(19) United States
(12) Patent Application Publication
(10) Pub. No.: US 2024/0110200 A1
(43) Pub. Date: Apr. 4, 2024

(54) Recombinant AAV Vectors and Gene Therapies for Treating Brain Diseases

(71) Applicant: City University of Hong Kong, Kowloon (HK)

(72) Inventors: Jufang He, Kowloon (HK); Ling He, Kowloon (HK); Heng Shi, Kowloon (HK); Yujie Yang, Kowloon (HK); Ge Zhang, Kowloon (HK); Xi Chen, Kowloon Tang (HK); Ezra Yoon, Kowloon (HK); Siuhin Lau, Kowloon (HK)

(21) Appl. No.: 17/935,698

(22) Filed: Sep. 27, 2022

(51) Int. Cl. C12N 15/86; A61P 25/00

(52) U.S. Cl. CPC C12N 15/86; A61P 25/00; C12N 2750/14143

(57) ABSTRACT

A recombinant AAV vector including a sequence for introducing the expression of G protein-coupled receptor 173 (GPR173) and specifically targeting GPR173 expressing neurons in a brain is provided. A method of restoring the excitatory/inhibitory (E/I) balance in brain or for prophylaxis and/or therapy of a neurological condition in a subject in need thereof by administrating the recombinant AAV vector or a GPR173 agonist to the subject is also provided.

The Sequence Listing file entitled “sequencelisting” having a size of 20,604 bytes and a creation date of Sep. 27, 2022, that was filed with the patent application is incorporated herein by reference in its entirety.

### FIELD OF THE INVENTION

The present invention generally relates to compositions and gene therapies for treating brain diseases by targeting a new receptor in the brain.

### BACKGROUND

Epilepsy is one of the most common neurological diseases and affects people of all ages, races, social classes, and geographical locations. In 2016, epilepsy accounted for more than 13 million disability-adjusted life-years (DALYs) and is responsible for 0.5% of the total disease burden. In children and young adults, epilepsy caused the most burden compared with any other neurological condition as estimated by the Global Burden of Diseases (GBD) study (Feigin, et al., Lancet Neurol. Vol. 18, pp. 459-480, 2019). Three-quarters of people living with epilepsy in low-income countries do not get the treatment they need. Approximately 30% of people with epilepsy will live with seizures during their lifetime without any therapeutic strategies. A study conducted by WHO and Health Action International in 46 lower middle-income countries assessed the availability, price, and affordability of five anti-seizure medicines-diazepam, phenytoin, phenobarbital, carbamazepine and valproic acid (Cameron, et al., Epilepsia, Vol 53 (6), pp. 962-969, 2012). The availability of generic oral anti-seizure medicines is less than 50% of the time in those countries. A lack of action to address the epilepsy treatment gap has dire consequences for people's lives and well-being and impacts social and economic development.

The International League against Epilepsy (ILAE) defines an epileptic seizure as “a transient occurrence of signs and/or symptoms due to abnormal excessive or synchronous neuronal activity in the brain”. There are fewer options in epilepsy therapy through glutamate receptors since the antagonists possess undesired side effects. In contrast, new interests in GABA_{A} receptor (GABA_{A}R) emerged in regulating neuronal excitability and treating epilepsy (Lasoń, W., et al., Pharmacol. Rep., Vol. 65, pp. 787-801, 2013). GABAergic neurons, accounting for 15˜20% of the neocortical neurons, keep the cortex balanced excitation and inhibition (Zhou, M., et al., Nat. Neurosci., Vol. 17, pp. 841-850, 2014). GABAergic neurons are further subcategorized into PV (39%), somatostatin (SOM, 23%), CCK (9%), vasoactive intestinal polypeptide (VIP, 11%), and other types (18%), based on their morphometric, transcriptomic features or neuropeptides they synthesized (Gonchar, Y., Front. Neuroanat., Vol. 1, pp. 1-11, 2008; Whissell, P. D., et al., Front. Neuroanat., Vol. 9, p. 124, 2015). Despite the role of CCK as a marker for the CCK-GABA neurons (Kubota, Y., et al., Brain Res., Vol. 752, pp. 175-83, 1997), its functional implication in GABA inhibitory synapses is still unknown.

Accordingly, the need exists for a composition, method, and therapy providing long-term medical efficiency of treating epilepsy to reduce the economic burdens. There remains a need to find new targets for developing drugs for treating brain conditions caused by imbalance of excitatory/inhibitory (E/I) in neurons with reduced toxicity and/or side effects.

### SUMMARY OF THE INVENTION

An embodiment of the present invention relates to a recombinant AAV vector including a sequence for introducing the expression of G protein-coupled receptor 173 (GPR173) and specifically targeting GPR173 expressing neurons in a brain.

An embodiment of the present invention relates to a method of restoring the excitatory/inhibitory (E/I) balance in brain by administrating an effective amount of a recombinant AAV vector or a GPR173 agonist to a subject in need thereof.

An embodiment of the present invention relates to a method of prophylaxis and/or therapy of a neurological condition in a subject in need thereof comprising the step of administrating an effective amount of a recombinant AAV vector or a GPR173 agonist to the subject.

Without intending to be limited by theory it is believed that the present invention provides an entirely new mechanism for treating E/I imbalance related diseases by targeting GPR173. It is believed that GPR173 would be a better target for drug development than GABA receptors. The vector, composition and therapy method according to the present invention target the whole brain and provide better solutions for patients with neurological/psychiatric problems in the global brain with fewer side effects than GABA receptors.

### BRIEF DESCRIPTION OF THE DRAWINGS

FIG. 1A shows injection of rAAV9-mDlx-DIO-ChR2-mCherry in the CCK-Cre and PV-Cre mice.

FIG. 1B shows the in vitro patch-clamp recording on the targeted pyramidal neurons (PC) while shining laser pulse to activate the infected CCK neurons (upper) and parvalbumin neurons (PV, lower); the confocal image depicting the morphology of a sampled neuron infused Alexa Flour 488 after patch-recording in the CCK-Cre mouse (scale bar: 15 μm); and neuronal firing responses to step currents.

FIG. 1C shows traces of IPSC responses to the laser pulse at 0.1 Hz from brain slices of CCK-Cre mice and PV-Cre mice, before and after the HFLS.

FIG. 2A shows the patch-clamp recording targeted at pyramidal neurons while shining a laser pulse to activate the infected neurons and perfusion of sulphated CCK8 (CCK8s) or aCSF; and neuronal response to step current, which is used to confirm that the recorded neurons are excitatory.

FIG. 2B shows IPSC to the laser pulse from brain slices of CCK-Cre mice with CCK8s or aCSF.

FIG. 3A shows rAAV9-mDlx-DIO-ChR2-mCherry-WPRE-pA (mDlx-DIO-ChR2-mCherry) and AAV9-mDlx-Cre-WPRE-pA (mDlx-Cre) injection into CCK-1/2R-KO mouse or CCK-KO mouse.

FIG. 3B shows the in vitro patch-clamp recordings on pyramidal neurons while shining laser pulse activated the infected GABAergic neurons in CCK-KO and CCK-1/2R-KO mice; and neuronal response to step current, which is used to confirm that the recorded neurons are excitatory.

FIG. 3C shows traces for the IPSC to the laser pulse from CCK-1/2R-KO mice (upper panel) and CCK-KO mice (lower panel) before and after the HFLS of the infected GABAergic neurons.

FIG. 4A shows a bar chart for the scores of similarities between GPCRs and known CCK receptors (CCK1R and CCK2R).

FIG. 4b shows cell surface binding assay for Flag-CCK2R, Flag-GPR173, and Flag-HTR1B with HA2A (negative control) or HA2A8s (scale bars, 10 μm); and bar chart of fluorescence intensity of Flag (GPCRs) or HA (binding ligands, N=33 to 40, one-way ANOVA with Tukey's post hoc test, ****P<0.0001).

FIG. 4C shows percentage identity correlation matrix of 7 orphan receptors with CCK1R and CCK2R.

FIG. 4D shows mDlx-DIO-ChR2-mCherry injection in the AC of CCK-Cre mice (left panel); immunochemistry staining of candidate receptors with CCK-GABA synapses (scale bars, 5 μm), and bar chart for the numbers of overlay dots per mm² (mCherry⁺, Synaptophysin⁺, and GPCR⁺, one-way ANOVA with Tukey's post hoc test, ***P<0.001).

FIG. 4E shows colocalized super-resolution image of mCherry and GPR173 (upper panel), and mCherry and GABA_{A}R (lower panel, scale bars: 1 μm, Pearson correlation coefficient (PCC) and Manders' overlap coefficient (MOC)).

FIG. 4F shows colocalized super-resolution image of GABA_{A}R and GPR173 (left panel, scale bar: 1 μm).

FIG. 4G shows value of PCC and MOC showing the colocalized possibility of mCherry with GPR173, mCherry with GABA_{A}R, and GABA_{A}R with GPR173. Data are expressed as mean±SEM.

FIG. 5A shows schematic illustration of the calcium imaging assay in the CHO-GPCRs cell lines.

FIG. 5B shows dose-dependent calcium response of CHO-GPR173 cells (left panel) and CHO-CCK2R cells (right panel) provoked by CCK8s; Relative Fluorescence Unit (RFU): the ratio of the RFU 30 s after (RFU_{t=30 s}) and the RFU before (RFUₜ₌₀) adding CCK8s/HHBS (one-way ANOVA with Tukey's post hoc test, *P<0.05, **P<0.01, ***P<0.001, ****P<0.0001).

FIG. 5C shows schematic illustration of the knockdown of the expression of GPR173 in the CHO-GPCRs cells by shRNA of GPR173.

FIG. 5D shows the calcium response of CHO-GPR173 cells provoked by 200 nM CCK8s between scramble or shRNA-infected CHO-GPR173 cells (one-way ANOVA with Tukey's post hoc test, *P<0.05).

FIG. 5E shows GPR173 mRNA expression level in a scramble or shRNA-infected CHO-GPR173 cells (Unpaired T-test; *P<0.05).

FIG. 6A shows schematic illustration of the gene therapy timeline used to inhibit intrahippocampal KA injection-induced epilepsy. After 3 weeks of intrahippocampal KA injection, 2 weeks of seizures observation are performed (Before). After intravenous viral (rAAV-CMV-GPR173-eGFP-PHP.eB or rAAV-CMV-eGFP-PHP.eB) delivery, the animals are continuously monitored for seizures for 8 weeks (After).

FIG. 6B shows scatter charts for the numbers of seizures from KA-induced chronic epileptic mice: Before (2 weeks) and After (8 weeks) the CMV-GPR173-eGFP-PHP.eB (left, N=8 mice) or CMV-eGFP-PHP.eB (right, N=8 mice) injection. Each dot represents one single seizure. The x-axis shows the whole observation period by weeks, and the y-axis shows the 24 hours in each day. Coordinates of each dot describe the exact time of each seizure onset.

FIG. 7A shows confocal images of eGFP and mCherry reporter gene expression with GPR173 in the mDlx-DIO-ChR2-mCherry and CMV-GPR173-eGFP injected mice (Upper panels), and mDlx-DIO-ChR2-mCherry and CMV-eGFP injected mice (lower panels; scale bars: 20 μm).

FIG. 7B shows averaged counts of eGFP, mCherry, and GPR173 triple-labeling of the two groups per mm² in the region of interest (ROI) (Upper panel; GPR173: n=9 sections; N=4 mice; Sham: n=9 sections; N=4 mice; one-way ANOVA with Tukey's post hoc test, ****P<0.0001); and averaged area (μm²) of eGFP, mCherry, and GPR173 triple-labelings of the two groups of ROI (mm2) (Lower panel; GPR173: n=9 sections; N=4 mice; Sham: n=9 sections; N=4 mice; one-way ANOVA with Tukey's post hoc test, ****P<0.0001).

FIG. 7C shows confocal images of eGFP reporter gene expression with GPR173 and GABA_{A}R in the cortex of CMV-GPR173-eGFP (upper panels) or the CMV-eGFP injected mice (lower panels, scale bars: 5 μm).

FIG. 7D shows percentage of eGFP, GPR173 and GABA_{A}R triple-labeling in the two groups (GPR173: n=12 sections, N=5 mice; Sham: n=9 sections, N=3 mice; one-way ANOVA with Tukey's post hoc test, ****P<0.0001). Data are expressed as mean±SEM.

FIG. 7E shows percentage of GPR173 and GABA_{A}R co-labeling in the two groups (GPR173: n=12 sections, N=5 mice; Sham: n=9 sections, N=3 mice; one-way ANOVA with Tukey's post hoc test, ****P<0.0001). Data are expressed as mean±SEM.

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

### Definitions

As used herein, “adeno-associated virus (AAV) vector” is a single-stranded DNA virus and belongs to the Dependovirus genus of the Parvoviridae family.

As used herein, “recombinant AAV vector” refers to a vector generated by replacing the wild type AAV open reading frames with a target (therapeutic or marker) gene expression cassette.

As used herein, “GPR173 expressing neurons” includes but is not limited to cortical excitatory neurons, inhibitory neurons, and other brain area excitatory neurons which express GPR173.

As used herein, “a gene enhancer sequence” refers to a DNA sequence that is distant from a gene but upregulate the gene's transcription.

As used herein, “inhibitory neurons” herein are neurons including but not limited to parvalbumin (PV), somatostatin (SOM), vasoactive intestinal peptide (VIP), cholecystokinin (CCK), neuropeptide Y (NPY), CBPs calbindin (CB), calretinin (CR).

As used herein, GABAergic neurons produce gamma-Aminobutyric acid (GABA), which is the main inhibitory neurotransmitter in the mammalian central nervous system (CNS). GABA is primarily synthesized from glutamate, catalyzed by glutamate decarboxylase (GAD), and is present at 30-40% of synapses. GABA induces either Cl− influx or K+ efflux, resulting in hyperpolarized neurons and reduced action potential. Dysfunction of GABA neurotransmission can result in several disorders, including schizophrenia and epilepsy.

As used herein, “pharmaceutically acceptable carrier” includes but is not limited to excipient, buffer, stabilizer or preservative that is used for pharmaceutical compositions and well known to those skilled in the art.

As used herein, “an effective amount” refers to the amount of the vector, the composition, or the GPR173-related therapeutics that is sufficient to increase the expression level of GPR173 to restore the E/I balance in brain.

### Long-Term Potentiation (LTP) of CCK-GABAergic Inhibition

GPR173 is a novel CCK receptor which locates within the CCK-GABAergic synapses. The release of CCK and GABA activate the postsynaptic GABA_{A}R and GPR173, leading to the enhancement of the inhibition. Only the release of GABA generates inhibition, but no potentiation of inhibition. LTP is the main form of synaptic plasticity reflecting the activity of synaptic information storage processes, and has been identified as the prime candidate to be the cellular correlate of learning and memory. In the epileptic mouse used in the examples, LTP may happens in several milliseconds to minutes. In the in vivo experiment of mice, the inhibition can last at least 1 hour, which commonly defined as long term. In some examples, in-vivo recording and in-vitro recording (see in vivo LFP and unit recordings and In vitro patch-clamp recordings described in the Example) are performed to test LTP.

Cholecystokinin (CCK) enables long-term potentiation of the excitatory circuit. The present invention relates to the regulation of CCK in the neuroplasticity of inhibitory synapses. Without intending to be limited by theory it is believed that activation of GABA neurons suppresses neuronal responses to the forthcoming auditory stimulus (AS) in the neocortex. High-frequency laser stimulation (HFLS) of GABAergic neurons potentiates this suppression in long term. HFLS of CCK-GABA neurons potentiates inhibitory postsynaptic current in the pyramidal neuron. The present invention surprisingly found that this potentiation is abolished in CCK-knockout (KO) mice but intact in mice with both CCK1R and CCK2R KO. The inventors further determine that G protein-coupled receptor 173 (GPR173) shows high similarities in the extramembrane structures with CCK1R and CCK2R and a high affinity to CCK8s and is co-localized with CCK-GABA synapse and GABA_{A} receptor. More importantly, the present invention surprisingly found exogenous upregulation of GPR173 suppresses seizure occurrence of epilepsy in mice. Accordingly, the present invention identifies GPR173 as a new CCK receptor mediating the potentiation of CCK-GABAergic inhibition, and develops vectors, compositions and methods of using GPR173 as therapeutic target for treating brain disorders related to excitation and inhibition imbalance.

### Vectors and Compositions

An embodiment of the present invention relates to a recombinant AAV vector that includes a sequence for introducing the expression of GPR173, and specifically targets the GPR173 expressing neurons in a brain.

The amino acid sequence of GPR173 is known in the art, and an embodiment of the instant invention includes all polynucleotide sequences encoding the hM3Dq. In some embodiment, GPR173 may include an amino sequence according to any of SED ID NOs. 1-2 or a functionally equivalent sequence with an identity of from at least about 80%, or at least about 90%, about 95%, about 96%, about 97%, about 98% or about 99% to about 100% of any one of SEQ ID NOs. 1-2.

Without intending to be limited by theory, it is believed that the vector of the present invention shows good blood brain barrier (BBB)-permeability and may result in more than doubling the GPR173 expression. It is believed that up-regulation of GPR173 may suppress epilepsy (for example, temporal lobe epilepsy (TLE), especially chronic TLE) even for a long term. Furthermore, no apparent adverse effects are observed in locomotion after the intervention using the inventive vector. In addition, it is believed that the up-regulation of GPR173 surprisingly brings a protective effect to cortical neurons from possible seizure-induced excitotoxicity.

In an embodiment herein, the recombinant AAV vector further includes PHP.eB. By including PHP.eB, the vector can penetrate blood-brain barrier (BBB) successfully to infects GABAergic neurons, and thus avoid the use of directly injection into brain tissues that may be too risky and cause undesired brain damages.

In an embodiment herein, the recombinant AAV vector further includes a gene enhancer sequence selected from the group consisting of CMV, Camkll, mDlx, GAD65, GAD67, and a combination thereof. It is believed that these gene enhancer sequences can help with upregulating the target sequence's expression and restricting the vector's expression in specific cell types.

In some embodiments, CMV may include a nucleic acid sequence according to any of SEQ ID NOs. 4-5 or a nucleic acid with an identity of from at least about 80%, or at least about 90%, about 95%, about 96%, about 97%, about 98% or about 99% to about 100% of any of SEQ ID NOs. 4-5.

In some embodiment, the mDlx enhancer may encode an amino acid sequence according to any of SEQ ID NOs. 6-9 or a functionally equivalent sequence with an identity of from at least about 80%, or at least about 90%, about 95%, about 96%, about 97%, about 98% or about 99% to about 100% of any one of SEQ ID NOs. 6-9.

In some embodiment, the Camkll enhancer may encode an amino acid sequence according to any of SEQ ID NOs. 10-14 or a functionally equivalent sequence with an identity of from at least about 80%, or at least about 90%, about 95%, about 96%, about 97%, about 98% or about 99% to about 100% of any one of SEQ ID NOs. 10-14.

In some embodiment, the GAD65 enhancer may encode an amino acid sequence according to SEQ ID NO. 15 or a functionally equivalent sequence with an identity of from at least about 80%, or at least about 90%, about 95%, about 96%, about 97%, about 98% or about 99% to about 100% of SEQ ID NO. 15.

In some embodiment, the GAD67 enhancer may encode an amino acid sequence according to SEQ ID NO. 16 or a functionally equivalent sequence with an identity of from at least about 80%, or at least about 90%, about 95%, about 96%, about 97%, about 98% or about 99% to about 100% of SEQ ID NO. 16.

In an embodiment herein, the sequence for introducing the expression of GPR173 in the recombinant AAV vector is a nucleic acid encoding any one of SEQ ID NOs. 1-2 and functional analogous thereof with an identity of at least about 80%, at least about 90%, at least about 95%, at least about 98%, or at least about 99% to about 100%, of any one of SEQ ID NOs. 1-2.

An embodiment of the present invention relates to a composition containing the recombinant AAV vector provided herein that includes a sequence for introducing the expression of G protein-coupled receptor 173 (GPR173).

GPR173 is believed to be a better target for drug development than GABA receptors as it mediates the enhancement of the GABAergic action rather than directly triggering the GABAergic inhibition. Without intending to be limited by theory, it is believed that manipulating GABAergic action directly will disrupt the basic function of GABAergic neurons. However, by upregulating GABAergic inhibition, the enhanced inhibition starts to show its significance when the excitation exceeds the threshold of a neuron. It is believed that the enhancement of the GABAergic action can be used in the treatment of brain diseases which are related with over-excitation or lacking inhibition effects. The present invention verifies the concept of enhancement of inhibition through GPR173. The present invention targets the whole brain due to numerous brain nuclei involved in the single seizure attack. The potentiation of inhibition through GPR173 leads to better drugs for patients with neurological/psychiatric problems in the global brain.

In an embodiment herein, the composition further includes a pharmaceutically acceptable carrier, as defined herein.

In some embodiments, the compositions described herein are designed for delivery to subjects in need thereof by any suitable route. In some embodiments, the composition may be in the form of solid, powder, drops, solution, etc.

### Uses and Therapy Methods

Provided herein is a use of GPR173 as a target for restoring the excitatory/inhibitory (E/I) balance in a brain; or a subject's brain; or a human brain, or for treating brain conditions related to E/I imbalance. For this purpose, the expression of GPR173 may be up-regulated with any one or any combination of the vector, the composition, and other GPR173-related therapeutics according to the invention.

In some embodiments, the excitatory/inhibitory (E/I) balance is restored by potentiating the inhibition of CCK-GABAergic inhibition for a long term.

The present invention provides an entirely new mechanism in strengthening the inhibitory effect on the postsynaptic neurons of CCK-GABA synapses by activating the GPR173. It is believed that CCK co-localizes in some VIP neurons in the neocortex. Similarly, to the CCK neurons, High Frequency Stimulation (HFS) is believed to induce VIP release leading to disinhibitory control in the cortex.

Without intending to be limited by theory it is believed that CCK-GABA neurons release CCK when they fire at high-frequency mode; the released CCK binds at the GPR173, inducing ischemic long-term-potentiation (iLTP) that strengthens their inhibition at the target neurons. The strengthened inhibition can bring back the balance between excitation and inhibition. The new finding of GPR173 mediated iLTP demonstrates a new mechanism of neuroplasticity of the inhibitory circuit.

Without intending to be limited by theory it is believed that GABA_{B}R may be used as a controller in a temporal integration manner for CCK release after high-frequency firings but not low-frequency firings from CCK-GABA neurons. Combining the current understanding with the new finding of GPR173, the present invention provides a new model for iLTP induction. It is believed that the activation of GPR173 by CCK release leads to calcium signaling either through opening the calcium channels in the postsynaptic neurons or releasing the calcium from the reticulum. GPR173 cell-line assay in the invention indicates CCK application triggers the calcium signal. The calcium signaling enables iLTP via potentially the following mechanisms 1) trafficking the reserved GABA_{A}R into the membrane, 2) turning on the silent GABA_{A}R, and 3) prolonging the action of GABA_{A}R and their related ion channels. A CCK2R-mediated astrocyte pathway is probably secondary to the above and supports/facilitates the iLTP. Astrocytes may release calcium to enhance the GPR173 signaling.

Without intending to be limited by theory it is believed that too much connectivity among the excitatory synapses in specific brain circuits leads to neurological/psychiatric problems, such as epilepsy and schizophrenia. In the epilepsy of whatever causes, genetic, cryptogenic, and others, there are too strong connectivities among the excitatory synapses, which spread the excitation, leading to an imbalance in the excitation and inhibition of the brain. Bringing back the balance of excitation and inhibition is the strategy for treating epilepsy.

Another embodiment of the present invention relates to a method of restoring the excitatory/inhibitory (E/I) balance in brain by administrating an effective amount of any one or any combination of the vector, the composition, and/or one or more GPR173-related therapeutics according to the invention to a subject in need thereof.

For example, an embodiment of the present invention relates to a method of restoring the excitatory/inhibitory (E/I) balance in brain by administrating an effective amount of the recombinant AAV vector herein or a GPR173 agonist to a subject in need thereof.

In an embodiment herein, the excitatory/inhibitory (E/I) balance in brain is restored by potentiating CCK-GABAergic inhibition with any one or any combination of the vector, the composition, and one or more GPR173-related therapeutics according to the invention. For example, in some embodiments, the method may comprise the steps of administrating the vector according to the invention, and the step of administrating one or more GPR173-related therapeutics, which are performed in any order or simultaneously. In some embodiments, the method may comprise the step of administrating the composition according to the invention, and the step of administrating the GPR173-related therapeutics, which are performed in any order or simultaneously.

In an embodiment herein, the GPR173 agonist is administrated at a dose of from about 1 pmol to about 100 mmol. In some embodiments, the GPR173 agonist is used at a low dosage of, for example, about 50 pmol, about 100 pmol, about 500 pmol, about 1 nmol, about 100 nmol, about 500 nmol, about 1 pmol, about 100 μmol, about 500 μmol, about 1 mmol, or about 50 mmol. Without intending to be limited by theory it is believed that CCK8s (a GPR173 agonist) can activate the GPR173 at nmol level.

In an embodiment herein, the recombinant AAV vector is administrated at a dose of from about 1E+5 vector genomes to about 1E+16 vector genomes (vg: vector genome), or from about from about 1E+9 vgs to about 1E+15 vgs. For example, the recombinant AAV vector is administrated at a dose of from 1E+5 vgs, about 1E+6 vgs, about 1E+7 vgs, about 1E+8 vgs, about 1E+9 vgs, about 1E+10 vgs, about 1E+11 vgs, about 1E+12 vgs, about 1E+13 vgs, about 1E+14 vgs, about 1E+15 vsg, or about 1E+16 vgs.

In an embodiment herein, the GPR173 agonist may be a CCK analogue. In some examples, the GPR173 agonist is selected from the group consisting of A-71623, CCK4, CCK8s and a combination thereof.

In an embodiment herein, the CCK-GABAergic inhibition is potentiated by upregulating GPR173 expression in neurons. In an embodiment herein, the up regulation of GPR173 expression means the expression level of GPR173 increases by at least about 5%, about 10%, about 15%, about 20%, about 25%, about 50%, about 60%, about 70%, about 80%, about 90%, about 100%, or even about 200%. As illustrated in Example 11, we compared the averaged size and numbers of GPR173 in same region and brain areas by immunochemistry between GPR173 upregulation group and control group to determine the increase percentages.

In an embodiment herein, the GPR173 expression is up-regulated in brain areas selected from the group consisting of cortex, hippocampus, amygdala, entorhinal cortex, and a combination thereof. It is believed that the excitatory neurons in these brain areas play a key role in many diseases, such as epilepsy, depression, schizophrenia, and autism.

In an embodiment herein, the composition of the invention is administrated by an administration method selected from the group consisting of an intramuscular injection, an intravenous injection, an intraperitoneal injection, a subcutaneous injection, orally taken, a spinal injection, an intraocular injection, and a combination thereof.

In an embodiment herein, one or more GPR173-related therapeutics according to the invention are selected from the group consisting of small peptide, agonist, nanoparticle, antibody, nucleic acid, mRNA, and a combination thereof. In an embodiment, the one or more GPR173-related therapeutics according to the invention are selected from the group consisting of small peptide, agonist, and a combination thereof. In some embodiments, the one or more GPR173-related therapeutics comprises a GPR173 agonist, preferably with high binding affinity and specificity so as to minimize side effects.

In an embodiment herein, the method of restoring the excitatory/inhibitory (E/I) balance induces long-term potentiation of CCK-GABAergic inhibition. In some embodiments, the potentiation of inhibition lasts at least about 3 days; about 1 week, about 2 weeks, about 3 weeks, about 1 month, about 2 months, about 3 months, or about 1 year, to at most about 5 years after the therapy. Due to the long-lasting potentiation of inhibition provided by the invention, the recombinant AAV vector and the composition of the invention can be administrated at long intervals, which is cost effective and may offer conveniences for patients who receive the vector or the composition. After one dose, the patients may be cured with less side effects compared with those patients who may need surgery or lifetime meditation.

Another embodiment of the present invention relates to a method of prophylaxis and/or therapy of a neurological condition in a subject in need thereof by targeting a GPR173 receptor in brains. As explained above, the GPR173 receptor may be a better target than GABA receptors as it mediates the enhancement of the GABAergic action rather than directly triggering the GABAergic inhibition.

An embodiment of the present invention relates to a method of prophylaxis and/or therapy of a neurological condition in a subject in need thereof by administrating an effective amount of any one or any combination of the vector, the composition, and other GPR173-related therapeutics according to the invention.

An embodiment of the present invention relates to a method of prophylaxis and/or therapy of a neurological condition in a subject in need thereof by administrating an effective amount of the recombinant AAV vector or a GPR173 agonist to the subject.

In an embodiment herein, the subject is administrated with only a single dose, or with multiple doses of the vector, of the composition or other GPR173-related therapeutics according to the invention. In an embodiment herein, the subject is administrated with only a single dose of the composition herein.

In an embodiment herein, the neurological condition is related to excitatory/inhibitory (E/I) imbalance, and particularly selected from the group consisting of epilepsy, autism, depression, schizophrenia, Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, a traumatic brain injury, and a combination thereof. Without intending to be limited by theory it is believed that these diseases are E/I imbalance related, and the recombinant AAV vector and therapy method according to the invention are capable of restoring such imbalance, which is beneficial for treating the above-listed diseases.

In an embodiment herein, the neurological condition is selected from the group consisting of epilepsy, autism, depression, schizophrenia, stroke, dementia, cancer, a traumatic brain injury, and a combination thereof; or from the group consisting of epilepsy, autism, depression, schizophrenia, and a combination thereof; or epilepsy. In an embodiment herein, epilepsy includes but is not limited to focal epilepsy, for example, selected from the group consisting of temporal lobe epilepsy, frontal lobe epilepsy, parietal lobe epilepsy, occipital lobe epilepsy, and a combination thereof.

In some embodiments, the subject involved in the use or the method according to the invention is selected from the group consisting of a mammal; or from the group consisting of a human; a non-human mammal, such as a dog, a primate, and a minipig, a cat; or a combination thereof.

### EXAMPLES

### Materials and Methods

### Animals

All procedures are done in accordance with guidelines approved by the Animal Subjects Ethics Sub-Committees of the City University of Hong Kong.

The following transgenic mice are utilized: Vgat-ires-Cre^{(slc32a1tm2(Cre)Low1}/J, Vgat-Cre, C57 background, Jackson Laboratory), mCCK-Conditional-KO (CCK-cKO, C57BL/6J background, Cyagen), Vgat-Cre-CCK-cKO (cross Vgat-Cre with CCK-cKO to specifically knock out CCK in GABAergic neurons), CCK-ires-Cre (Cck^{tm1.1(Cre)Zjh}/J, C57BL/6J background, for short CCK-Cre, Jackson Laboratory), PV-ires-Cre (Pvalb^{tm1(cre)Arbr}/J, PV-Cre, C57BL/6J background, Jackson Laboratory), CCK-CreER (Cck^{tm2.1(Cre/ERT2)Zjh}/J, CCK-KO, C57BL/6J background, Jackson Laboratory), CCK-AR/BR-KO (Cckar^{tm1Kpn} Cckbr^{tm1Kpn}/J CCK1R/2R-KO, 129S1 background, Jackson Laboratory) and C57BL/6J mice. The standard procedures and the recommended primers for genotyping are adopted. The primer pairs used for each mouse line are listed in the Table 1.

### Generation of Vgat-Cre-CCK-cKO Mice

Homozygous male Vgat-Cre mice (+/+) are crossed with homozygous female CCK-cKO (+/+) mice for the first generation of hemizygous Vgat-Cre (+/−)-CCK-cKO (+/−) mice. Vgat-Cre (+/+)-CCK-cKO (+/+) is obtained after mixing male and female hemizygous Vgat-Cre (+/−)-CCK-cKO (+/−) mice.

### Local Virus Injection in the Auditory Cortex

The mice (6-8 weeks old) are injected with atropine (0.05 mg/kg, Intramuscular injection (i.m.), Sigma-Aldrich, www.sigmaaldrich.com/, St. Louis, MO) to inhibit tracheal secretions before the anaesthetics (pentobarbital sodium, 80 mg/kg, Dorminal 20%, Alfasan, Woerden-Holland) intraperitoneally (i.p.). The body temperature is maintained between 37-38° C. with a heating pad (RWD Life Science, https://www.redls.com/, Shenzhen, China). A local anaesthetic (xylocaine, 2%) is also applied to the incision site for analgesia. Craniotomy of two holes of 300-μm diameter is performed on the head-fixed mice to access their AC (−2.0 to −3.0 mm posterior to the bregma and −4.0 to −4.3 mm to the midline) on a stereotactic device (RWD Life Science). A double loxP-flanked (DIO) Cre-dependent adeno-associated viral (AAV) vector expressing channelrhodopsin-2 variants (ChETA) fused with an enhanced yellow fluorescent protein (eYFP, AAV-EF1a-DIO-ChETA-eYFP, 8E+12 vg/mL, vg: vector genome; University of North Carolina Vector Core, Wilmington, North Carolina, US, https://www.ncaa.com/schools/north-carolina) is injected into the ACs (300 nL of the AAV vector at 30 nL/min at 4 sites, with Nanoliter Injector (World Precision Instruments; UK; https://www.wpiinc.com/) of the Vgat-Cre mice or the Vgat-Cre-CCK-cKO mice.

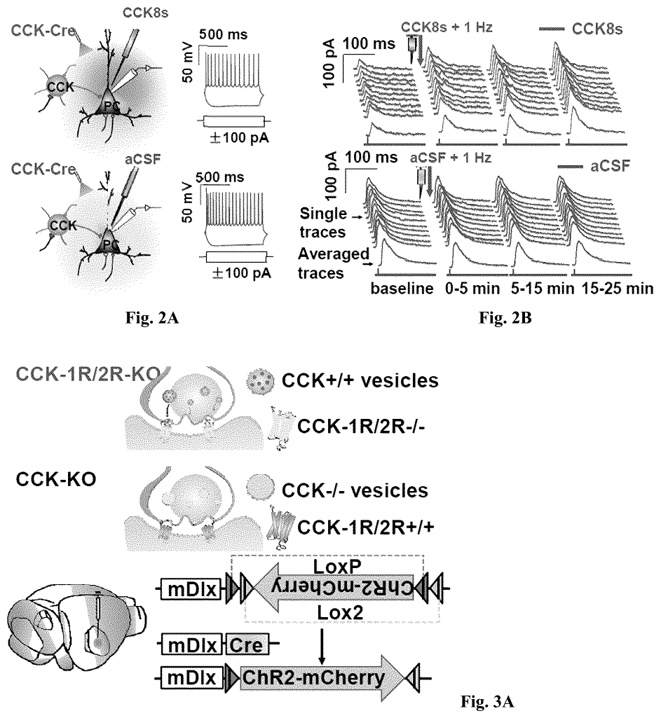

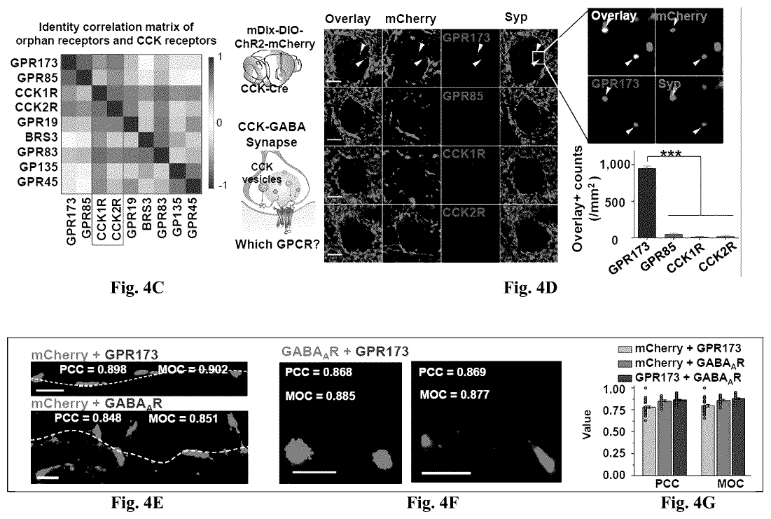

For the experiments in FIG. 2, and FIGS. 4D-4G, rAAV9-mDlx-DIO-ChR2-mCherry-WPRE-pA(5.18E+12 vg/mL; BrainVTA, http://www.brainvta.com/, Wuhan, China) is injected in the AC of CCK-Cre and PV-Cre mice.

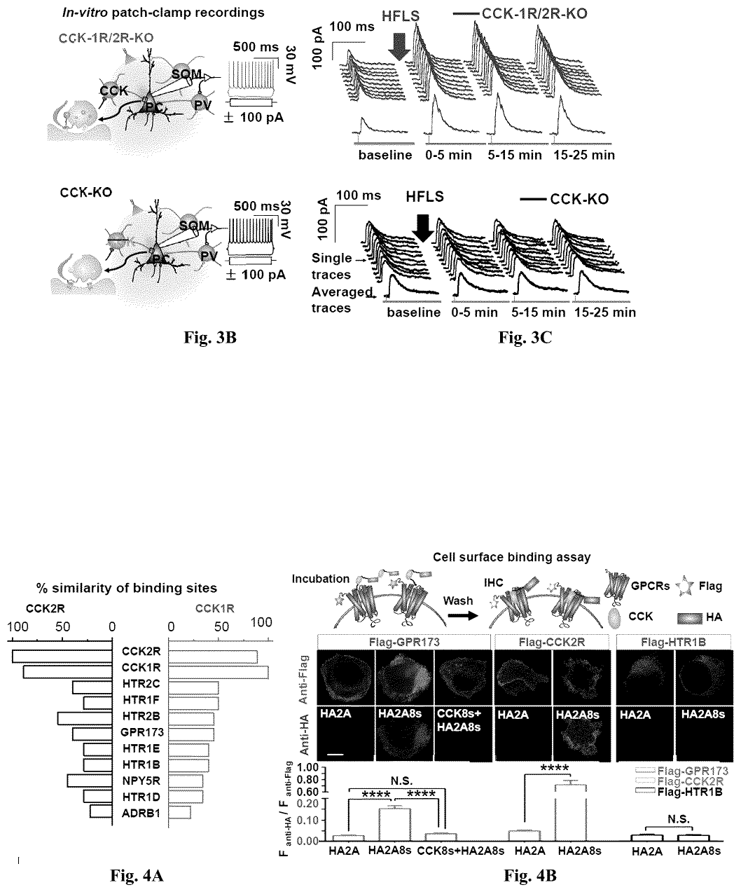

For the experiment in FIG. 3, a mixture of AAV9-mDlx-Cre-WPRE-pA (1.42 E+12 vg/mL; BrainVTA) and rAAV9-mDlx-DIO-ChR2-mCherry-WPRE-pA (2.09 E+12 vg/mL) is injected into the AC of CCK1R/2R-KO and CCK-KO mice.

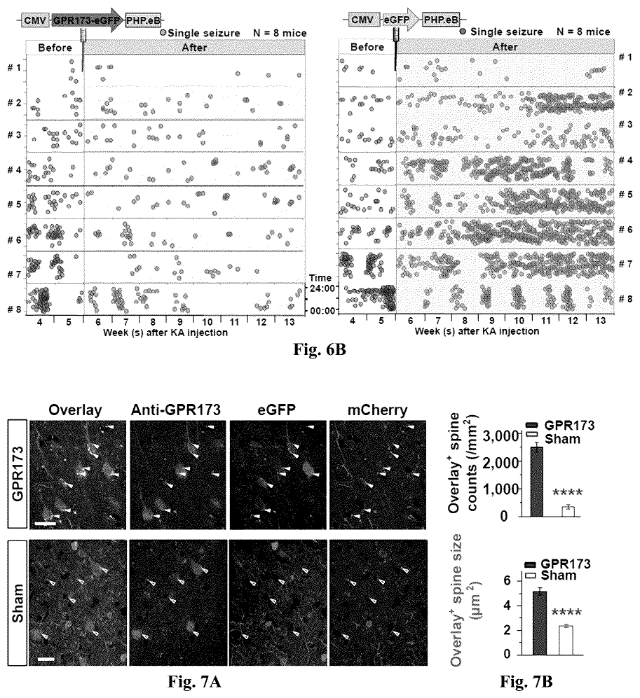

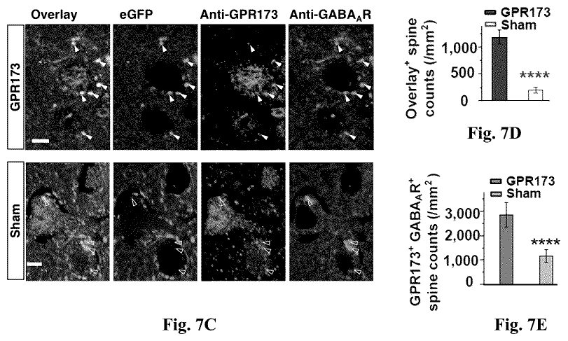

For the experiment in FIGS. 6 and 7, a blood-brain barrier (BBB)-penetrating AAV-PHP.eB is designed and then produced in BrainVTA. The AAV includes a woodchuck hepatitis virus post-transcriptional regulatory element (WPRE) and expresses an enhanced green fluorescent protein (eGFP) and orphan GPCR173 under the control of the neuron-specific enhancer sequence CMV (rAAV-CMV-GPR173-eGFP-PHP.eB or rAAV-CMV-eGFP-PHP.eB). The virus is injected into the transverse venous sinus under the same procedure for brain injection.

For the experiment in FIGS. 7A-7B, a cocktail of rAAV9-mDlx-DIO-ChR2-mCherry-WPRE-pA (2.09 E+12 vg/mL; BrainVTA) and rAAV-CMV-GPR173-eGFP-PHP.eB (1.01 E+12 vg/mL) are injected in the cortex of one group CCK-Cre mice, while a cocktail of rAAV9-mDlx-DIO-ChR2-mCherry-WPRE-pA and rAAV-CMV-eGFP-PHP.eB (1.06 E+12 vg/mL) in the other group mice.

### AAV-PHP.eB Production

The viral tools are all packaged by BrainVTA (BrainVTA Co., Ltd., Wuhan, China, http://brainvta.bioon.com.cn). Briefly, the main pAAV plasmid contains AAV2 ITRs. The AAV2 comprises a nucleic acid sequence according to SEQ ID NO. 3 or a nucleic acid with an identity of from at least about 80%, or at least about 90%, about 95%, about 96%, about 97%, about 98% or about 99% to about 100% of SEQ ID NO. 3. Separate Rep/Cap plasmid and the helper Plasmid provides components of the viral replication machinery and the capsid proteins of selected AAV serotypes. After HEK293 cell lysis, viral particles are purified by CsCl gradient ultracentrifugation. And rAAVs are tittered by quantitative polymerase chain reaction (qPCR).

### Optogenetics: In Vivo LFP and Unit Recordings

### In Vivo LFP Recordings

In vivo recordings is performed at weeks 3-4 after the virus injection, as described in the following articles: Li, X., et al., Cholecystokinin from the entorhinal cortex enables neural plasticity in the auditory cortex, Cell Res., Vol. 24, pp. 307-330, 2014; Zhang, Z., et al., Visuoauditory Associative Memory Established with Cholecystokinin Under Anesthesia Is Retrieved in Behavioral Contexts, J. Neurosci., Vol. 40, pp. 2025-2037, 2020; and Chen, X., et al., Cholecystokinin release triggered by NMDA receptors produces LTP and sound-sound associative memory, Proc. Natl. Acad. Sci., Vol. 116, pp. 6397-6406, 2019. Briefly, the mice are injected with atropine to inhibit tracheal secretions 15 mins before the anaesthetics with urethane sodium (2 g/kg, i.p., Sigma-Aldrich, St. Louis, MO, USA; https://www.sigmaaldrich.cn/) and the craniotomy is performed to access the AC (−1.8 to −3.5 mm posterior to the bregma and −3.5 to −4.5 mm to the midline).

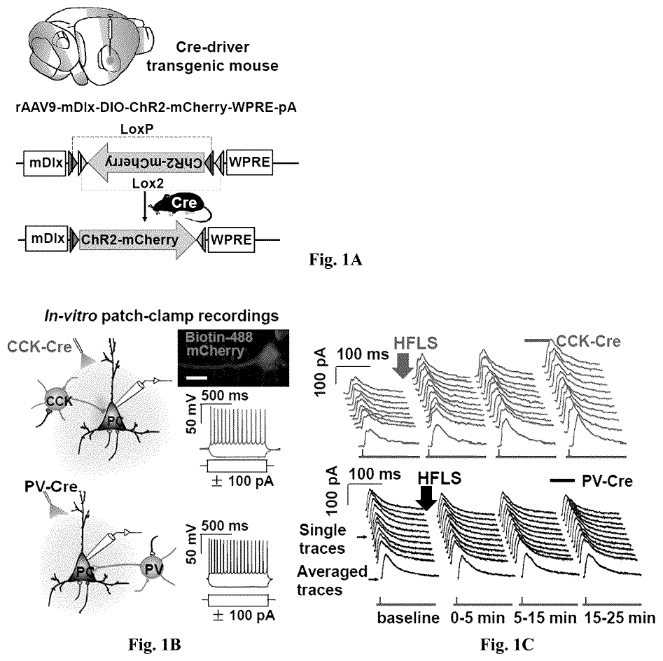

A recording borosilicate glass (WPI, World Precision Instruments; UK; https://www.wpiinc.com/) electrode array (pulled with a Sutter-87 puller, Sutter Instrument; CA, US, https://www.sutter.com/)) is inserted in 300-500 μm deep and a laser fiber (RWD Life Science, with 200 μm diameter) is placed on the surface of the AC where the virus is infused. To advance the electrode array, a stepping-motor micro driver is controlled from outside the soundproofed chamber. The signals are amplified using the TDT workstation (OpenEX, TDT, Alachua, Florida, USA; https://www.tdt.com/). The TDT workstation generates a noise burst of 100-ms duration that is led to a coupled electrostatic speaker as the auditory stimulus (AS, 50-105 dB, Sound Pressure Level, SPL). The sound intensity is measured and adjusted by a condenser microphone (Center Technology, Taipei; http://www.centertek.com) when it reaches the animal. A laser generator (NEWDOON, Changchun, China; http://www.newdoon.com/) delivers a laser pulse (5 ms duration) at a 473-nm wavelength. The intensity of the laser is set at around 0 to 10 mW at the end of the laser fiber in FIGS. 1-3, and FIGS. S1-S7.

The input-output curve between the sound intensity (60-95 dB, SPL) and the amplitude of the LFP is measured and the intensity that evoked 50% of the maximum response is adopted as the sound intensity. The input-output curve between the laser power (0-15 mW) and the amplitude of the LFP is also measured to help select the laser power in FIG. 1 and FIG. S2A. The laser power that evoked 50% to 70% of the maximum response is adopted as the stimulation power in every experiment.

The viral expression is confirmed by examining the laser-induced suppressive effect on the spontaneous neuronal response in FIG. S3A. The interval between the laser and AS is selected to achieve the optimal suppressive effect of the laser-activated inhibitory neurons onto the neuronal response to the forthcoming AS. A gap of 50 ms is set for subsequent experiments. The LFP traces are calculated with Matlab (MathWorks.Inc, Natick, US; https://ww2.mathworks.cn/) and plotted over time.

The spontaneous neuronal activities and responses to the laser and AS combination as units and LFP (0.1 Hz) are examined for 15 min (FIG. 1, FIGS. S5 and S7). Single-pulse laser (3-5 mW, 5-ms duration) in 0.1-Hz repetitions is delivered 50 ms before the AS and recorded for 15 min (baseline) and 60 min after the HFLS (the middle panels in the FIG. 1d and FIG. 1e, 5 pulses at 40 Hz, repeated in 40 trials at 0.1 Hz). The frequency of HFLS is examined as a parameter to confirm the laser-evoked response, which helps to select the laser frequency as the desired HFLS and remove the light/heat effects (FIG. S2B, 1-120 Hz, repeated in 20 trials at 0.1 Hz).

### In Vivo LFP Analysis

The LFP amplitudes are averaged every six trials (MATLAB, MathWorks.Inc, Natick, US; https://ww2.mathworks.cn/), representing a 1-min recording and the representative waveforms are plotted with MATLAB.

Group data are presented as the mean±standard error of the mean (SEM). 3 to 10 mice per condition for each experiment are used. Male or female mice are randomly selected for each experiment. For all in vivo electrophysiology experiments, “n” refers to the number of recording sites per condition, and “N” refers to the number of animals. The data are analyzed in a blinded manner. A two-way analysis of variance (ANOVA) and Tukey's post hoc test are performed for the examples involving two independent variables by SPSS 28.0.1 (Chicago, US, http://www.spss.com.cn) or Excel, assuming a normal distribution where n is too small to determine distribution. Origin Pro 8.5 is used to show the group data in all figures.

### Unit Waveform Processing

Commercial hardware and software (OpenEX, TDT, Florida, United States, https://www.tdt.com/) are used to analyze the waveform from every unit with Openbridge (TDT, Florida, United States, https://www.tdt.com/), offline sorter Plexon (Plexon, Dallas, Texas, US, https://plexon.com), and Neuroexplorer (Nex Technologies, Colorado Spring, US; http://www.neuroexplorer.com). Multi-units are recorded primarily with the current electrode preparation.

The inventors discriminate units from the PLX file against principal component analysis using the Plexon Offline Sorter (Plexon, Dallas, Texas, US, https://plexon.com). Two criteria are adopted to ensure the quality of recorded units: 1) the signal larger than three times the noise band; and 2) no more than 1% of the inter-spike intervals shorter than 2 ms. Only those neurons with at least 1000 detected spikes within 15 mins are considered in the analysis.

The sorting is opened in the NEX5 file by NeuroExplorer. Rasters and histograms for units are plotted using Perievent rasters (Neuroexplorer; Nex Technologies, Colorado Spring, US; http://www.neuroexplorer.com) with specific, fixed reference events (laser, AS, or sweep) with 5-ms bin width. The single and averaged waveforms are extracted into Origin Pro 8.5 (OriginLab, Northampton, Massachusetts, US, https://www.originlab.com). Counts per bin (5 ms), depending on the duration of the fixed reference event time for a comparable experiment, are used for firing rate quantification.

The average firing rate during the 100-ms AS presentation are counted. The period between the laser and 100 ms after the AS presentation in the spontaneous firing rate quantification is excluded because such firing corresponded to the indirect activation of GABAergic neurons or a delayed noise response.

### In Vitro Patch-Clamp Recordings

### Mouse Brain Slice Preparation

In the experiments of FIGS. 2-3, a protective cutting and recovery method is adopted in preparing acute brain slices of CCK-Cre, PV-Cre, CCK-KO, and CCK1R/2R-KO mice to achieve a higher success rate for patch-clamp, as previously described in Chen, X., et al., Natl. Acad. Sci., Vol. 116, pp. 2019. Briefly, the anaesthetized mice are perfused transcardially with NMDG-aCSF (92 mM NMDG, 2.5 mM KCl, 1.25 mM NaH₂PO₄, 30 mM NaHCO₃, 20 mM HEPES, 25 mM glucose, 2 mM thiourea, 5 mM Na-ascorbate, 3 mM Na-pyruvate, 0.5 mM CaCl₂·4H₂O, and 10 mM MgSO₄·7H₂O; pH 7.3-7.4), and the brain is extracted and cut into 300 μm-thick sections. The slices are transferred to NMDG-aCSF for 10 min at 32-34° C. to allow protective recovery and then to room-temperature HEPES-aCSF (92 mM NaCl, 2.5 mM KCl, 1.25 mM NaH₂PO₄, 30 mM NaHCO₃, 20 mM HEPES, 25 mM glucose, 2 mM thiourea, 5 mM Na-ascorbate, 3 mM Na-pyruvate, 2 mM CaCl₂·4H₂O, and 2 mM MgSO₄·7H₂O; pH 7.3-7.4) for at least 1 h before recording.

### Whole-Cell Recordings

Whole-cell recordings (with Multiclamp 700B amplifier and Digital 1440A digitizer, Molecular Devices, www.moleculardevices.com, Menlo Park, California, USA), targeting the AC of the brain slices in room-temperature aCSF (119 mM NaCl, 2.5 mM KCl, 1.25 mM NaH₂PO₄, 24 mM NaHCO₃, 12.5 mM glucose, 2 mM CaCl₂·4H₂O and 2 mM MgSO₄·7H₂O, ˜25° C.) are performed. Patch pipettes are pulled with resistance between 3-5 MQ from borosilicate glass (WPI, World Precision Instruments; UK; httpss://www.wpiinc.com/) on a Sutter-87 puller (Sutter Instrument; CA, US, https://www.sutter.com/). The intracellular solution contains: 145 mM K-Gluconate, 10 mM HEPES, 1 mM EGTA, 2 mM Mg-ATP, 0.3 mM Na₂-GTP, and 2 mM MgCl₂; pH 7.3; 290-300 mOsm. Similarly, after Giga-Ohm seal formation, recordings are made and then terminated if Rs changes more than 20%. Pyramidal neurons are selected based on the pyramidal-like shape and the firing pattern of regular spiking by injecting step currents. Biotin-Alexa 488 (A12924, Thermo Fisher, Waltham, MA, US, https://www.thermofisher.cn) is infused after recording to confirm the patched pyramidal neuron.

Laser stimulation is applied through an optical fiber placed ˜200 μm from the recording neuron using Aurora-220 (473 nm, NEWDOON, Changchun, China; http://www.newdoon.com/) to activate ChR2⁺ neurons (GABAergic). In the examples, only neurons that respond to the laser pulse is recruited. HFLS (10-pulse burst at 40 Hz, repeated for 10 trials) or Low-frequency Laser Stimulation (LFLS, 1 Hz, 100 pulses) with a 15-s interval is delivered. Inhibitory postsynaptic currents (IPSCs) to the laser stimulation (1 pulse per 15 s, 5 ms in duration) under voltage-clamp recording mode (holding at −60 mV to −50 mV) are recorded for 5 min as the baseline before and for 25 min after the HFLS or LFLS.

In a subset of examples, 200-nM CCK8s is applied via bath application 3 min before the LFLS (FIG. 2).

For the normalization of recorded IPSC in the CCK-Cre, PV-Cre, CCK-KO, and CCK1R/2R-KO mice, only the patched neurons are recruited when the IPSC is greater than 35 pA.

### Searching Potential Candidates of CCK Receptors by Bioinformatics Methods

Proteomics Screen Out in the G Protein-Coupled Receptors (GPCRs) Pool, in Comparison with CCK1R, CCK2R

Approximately 200 GPCRs, both orphans and characterized receptors, known from the literature, as FASTA formatted sequences obtained from UniProt and available at https://www.uniprot.org are downloaded. All the FASTA formatted sequences are merged into one word file for further analysis. For the hierarchical cluster and scores for CCK1/2R, the two parameters as follows are set: 1) the binding sites from GPCRs' secondary structure (extra-membrane) are selected based on the CCK binding domains of CCK1R and CCK2R; and 2) the amino acid sequences are compared based on the hamming distance. After identifying all proteins with these two parameters, those GPCRs that had both high scores of similarities with CCK1R and CCK2R are selected. An online free software NetsurfP ver1.1 (new version available at http://www.cbs.dtu.dk/services/NetSurfP-1.1/) is used for analysis. NetsurfP predicts amino acids' surface accessibility and secondary structure in an amino acid sequence. Example 5 validates total scores of similarities with CCK1R and CCK2R above 30 without any additional manual inspection in FIG. 4A.

### Percentage Identity of the CCK1R, CCK2R, and 79 Orphan GPCRs

The percent identity matrix are examined for 79 orphan GPCRs with CCK1R and CCK2R. Percentage identity defines the quantitative correlation estimation between two sets of sequences. Closely correlated species have a higher percentage identity for a given amino acid sequence. Hence percent identity reflects the relatedness to some extent. The sequences of 79 orphan GPCRs, together with CCK1R and CCK2R proteins, are analyzed and plotted by a self-designed program in FIG. 4C.

We used Clustal Omega (http://www.clustal.org/omega/) to extract the homologous information of the 79 orphan receptors from class A GPCRs, aligned with 2 known CCK receptors. The percentage identity correlation score defines the quantitative correlation estimation between two sets of sequences. According to the percentage identity correlation score among 79 orphan receptors and CCK1R/2R, we select top-scored orphan receptors normalized by CCK1R and CCK2R for anatomical examination.

### Peptide Synthesis

Peptides and modified peptides are chemically synthesized (by Bank Peptide Biotechnology, Synpeptide or Shanghai Ke Biochem Co., Ltd.; Shanghai, China). HPLC analysis results ensure that the purity of peptides is over 97%. The peptides and modified peptides sequences are listed in Table 1.

### Cell Surface Binding Assay

HEK293T cells are transfected with pCDNA3.1(+)-Flag-GPCR (purchased from Public Protein/Plasmid Library, Nanjing, China, http://ppl.biogot.com; or home-made by us based on pCDNA3.1(+) backbone). The cells are incubated with HA-ε-Ahx-ε-Ahx or HA-(ε-Ahx)₂-CCK8s dissolved in HHBS solution (Hank's solution including 20 mM N-2-hydroxyethylpiperazine-N′-2-ethanesulfonic acid (HEPES), pH=7.4) for 30 min at room temperature, 48 h after transfection by Lipo6000 (Beyotime Biotechnology, Shanghai, China, https://www.beyotime.com/). After three washes with HHBS, cells are fixed in 4% paraformaldehyde and 4% sucrose in PBS for 15 min at room temperature. Cells are blocked in 5% normal goat serum in PBS, and staining is performed in detergent-free conditions without cell permeabilization after three washes by PBS. Primary antibody rabbit anti-HA (CST, 3724 Boston, US, https://www.cellsignal.com), anti-GPR173 (PA5-50976, ThermoFisher, Waltham, MA, US, https://www.thermofisher.cn) and mouse anti-Flag (CST-8146, Boston, US, https://www.cellsignal.com) are used to detect HA and Flag tags. For binding assay of pCDNA3.1(+)-AcGFP-CCK1R, only rabbit anti-HAprimary antibody is used to detectHAtag. Images are captured on a Leica SP8 confocal microscope (Leica Microsystems, Danaher Corporation, US, https://www.leica-microsystems.com/). Images within each experiment are collected using identical laser-power, offset, and gain.

### Immunohistochemical Staining

For immunohistochemistry, mice are deeply anaesthetized by an overdose of pentobarbital sodium. Mice are transcardially perfused with 30 mL cold phosphate-buffered saline (PBS) and 30 mL 4% (w/v) paraformaldehyde (4% PFA). Brain tissue is removed, post-fixed with 4% PFA, and treated with 30% (w/v) sucrose in 4% PFA at 4° C. for 2˜3 days. Brain tissue is sectioned on a cryostat (30 μm for standard staining and 10 μm for Super-Resolution imaging) (Leica CM3500, Germany) and preserved with antifreeze buffer (20% (v/v) glycerine and 30% (v/v) ethylene glycol diluted in PBS) at −20° C. For immunostaining and statistics of co-labelled neurons and axonal synapses in the AC, serial sections containing the AC are selected for analysis. Brain sections are rinsed three times with PBS and blocked with blocking buffer (10% (v/v) goat serum in PBS with 0.2% (v/v) Triton X-100) for 2 h at room temperature. Sections are incubated with the primary antibody (see, Table 1) at 4° C. for 24-36 h. After washing 4 times in PBS (each time for 10 min), sections are incubated with the corresponding fluorophore-conjugated secondary antibodies (see, Table 1) for 2.5-3 h at 25° C. Sections are then rinsed with PBS three times before DAPI staining (1:5000 (v/v) diluted in PBS) or mounting. All the sections are mounted with 70% (v/v) glycerine in PBS (standard staining) or imaging buffer (Super-Resolution imaging) on slides.

### Image Acquisition and Analysis

Image acquisition (10×, 20×, 63×, and 100× magnification) is performed by Nikon A1hD25 confocal Microscope (Nikon, Japan). The confocal microscope is equipped with a time delay integration camera and performed line scanning that offers fast acquisition at a high resolution of the fluorescent signal.

Images are took at 20× magnification and montaged together with the same laser settings between the parallel studies. The gain and exposure parameters are optimized for each image. For the magnified images showing the labelling details, z stacks images throughout the tissue are captured. For quantification of co-localization, neurons expressing the indicated reporter are counted using only the corresponding color channel. Then, among these cells, the number of neurons co-expressing the marker of interest is counted by the Nikon imaging analysis software (NIS-Elements Viewer 5.21). The bright spot detection is performed in the fields of interest brain regions, with the software under an appropriate fluorescent threshold. A neuron is positive for a given marker if the corresponding signal is above background fluorescence. The ratio of neurons co-expressing both markers over the total number of cells expressing only the reporter is then calculated.

The ‘n’ presented in the Figures show the representative biological replicates, ‘N’ indicated independent animals. At least 3 biological replicates per data point are included for all quantifications used in the examples.

### Super-Resolution Imaging and Processing

The staining process is described as immunohistochemistry (IHC) staining. The difference is the secondary antibody and mounting. Two fluorophores are used (Alexa Fluor 647 in combination with Alexa Fluor 790) as the secondary antibody (Table 1) in the super-resolution imaging slices processing. To immobilize the labelled slice, 200 μL fluorescent beads (Thermo Fisher, Waltham, MA, US, https://www.thermofisher.cn) are firstly applied onto the 1.5 μm-thickness cover glass with 120° C. heating to tightly attach. After several washes with ddH₂O, a well-labelled slice of AC is gently attached to the cover glass where beads attached. After drying up, the slide is used to seal the slice with imaging buffer (150 mM Tris, pH 8, 1% glycerol, 1% glucose, 10 mM NaCl, 1% β-mercaptoethanol, 0.5 mg/ml glucose oxidase, 40 μg/ml catalase). Direct Stochastic Optical Reconstruction Microscopy (dSTORM) imaging is performed in a freshly prepared imaging buffer, which can slow down the speed of fluorophores quenching during imaging acquiring. dSTORM imaging is performed on a 20× and 100× Olympus microscope (NanoBiolmaging SRiS 2.0 (STORM) Super-Resolution Microscope, HKU) (Olympus, Japan) and analyzed with the ImageJ (NIH, Bethesda, US, https://iadrp.nia.nih.gov).

In super-resolution capture mode, slice focus is firstly locked with NBI Rohdea 3.0 under 20× through capturing the signal of fluorescent beads attached on slides. A Z-stack of fluorescent beads at this moment acts as a reference. At first, a 405 nm laser is turned on to activate the two fluorophores. Under the illumination at 10%-15% of the maximum output power of 656 nm and 750 nm laser, a preview of the target signal is observed before imaging. 3,000 frames with 656 nm and 750 nm laser illumination at 30%-50% of maximum output power to activate the two fluorophores are acquired. Drift are estimated using the inbuilt function in SRiS 2.0 and correction is applied during analysis. The raw data and single-channel Excel file, and raw data files are auto-generated for analysis after capturing.

The Excel file is directly imported into ImageJ. After Dataset Reconstruction with the same parameter of two channels, red and green RGB colors are used to differentiate the two fluorophores of two channels and these two colors are merged into one channel, which forms the overlay images as shown in FIGS. 4E-4G.

Pearson's correlation coefficient (PCC) is a common metric to measure the predictability of co-localization (Aaron, J. S., et al., J. Cell Sci., Vol. 131, pp. 1-10, 2018). In more mathematical terms, the PCC can be thought of as the covariance between the two images, normalized by the product of their standard deviations.

### Generation of GPCRs Overexpression-Cell Line

HEK293T cells are maintained in DMEM/High glucose medium (Thermo Fisher, Waltham, MA, US, https://www.thermofisher.cn), supplemented with 10% fetal bovine serum (Natocor-Industria Biolóica), 1% Non-Essential Amino Acids (NEAA, Gibco, Thermo Fisher, Waltham, MA, US, https://www.thermofisher.cn), and 1% Glutamax (Gibco, Thermo Fisher, Waltham, MA, US, https://www.thermofisher.cn) at 37° C. in a humidified atmosphere of 5% CO₂. Chinese hamster ovary (CHO) cells and infected CHO cells (CHO-GPCRs) are cultivated at 37° C. in a humidified atmosphere with 5% CO₂ in DMEM/F-12 medium (Thermo Fisher, Waltham, MA, US, https://www.thermofisher.cn, supplemented with 10% fetal bovine serum (Natocor-Industria Biológica, Carlos Pass in Corvado, Argentina). To obtain lentivirus, the HEK293T cells are seeded for 70%-90% confluency in 6-well tissue culture plates and incubated for 10-12 h in the mixture of calcium phosphate transfection reagent (31.25 μl 2 M CaCl₂) solution, 250 μl 2×HBS (0.05 mol/L HEPES, 0.012 mol/L D-(+)-Glucose, 0.28 mol/L NaCl, 0.023 mol/L KCl, 0.0015 mol/L Na₂HPO₄, 218.75 μl H₂O) and 1 ml fresh medium, with 4 μg pLVX-puro-GPCR plasmids (Public Protein/Plasmid Library, Nanjing, China, http://www.geneppl.com), 3 μg pSPAX2 plasmid (Addgene, plasmid #12260, Cambridge, Mass, US, https://www.addgene.org) and 1.2 μg pMD2.G plasmid (Addgene, Cambridge, Mass, US, https://www.addgene.org, plasmid #12259). The cells are cultured for an additional 36 h in their normal medium before collecting virus supernatant and infecting CHO cells.

Meanwhile, around 1×10⁶ CHO cells are seeded in 6-well plates, each well for 12 h, 0.5 mL virus supernatant, and 0.1% polybrene (Sigma-Aldrich, St. Louis, MO, USA; https://www.sigmaaldrich.cn/) is mixed and added in 1 mL fresh normal medium. The second infection is performed for 24 h, and the virus supernatant mix is replaced with the fresh normal medium of CHO after 6-8 h of each infection. After 2 days of the second infection, puromycin (Thermo Fisher, Waltham, MA, US, https://www.thermofisher.cn) is added at the final concentration of 10 μg/mL for one week. To obtain high expression cell lines, single-cell is isolated by limited dilution in 96-well plates and RT-qPCR is performed after one month's culturing. Finally, 1-2 high expression cell lines are selected for further characterization.

RT-PCR and Detection of mRNA Expression of CCK JR, CCK2R, and GPR173

The RNA simple Total RNA Kit (TIANGEN Biotech, http://www.tiangen.com, Beijing, China) is used to isolate total RNA from the cells and 1.6 μg RNA and Fast King RT Kit (TIANGEN Biotech, http://www.tiangen.com, Beijing, China) are selected to generate cDNA. The RT products are amplified by PCR using the following primers:

cck1r (183 bp):
GGTCATCACGGTGCTGATTC/CCCATGAAGTAGGTGGTGGT
cck2r (186 bp):
CTGGGGAAGACAGTGATGGT/GCAGCCAACACAGGAAGAAA
gpr173 (220 bp):
TCTGGTCACCCTACATCGTG/CAGTAGGGTTCTCTGGGAGC
actb (206 bp):
CCTCTATGCCAACACAGTGC/CCTGCTTGCTGATCCACATC

PCR products are separated on 1% agarose gels and visualized by GeneGreen Nucleic Acid Dye (TIANGEN Biotech, http://www.tiangen.com, Beijing, China).

GPR173 Knockdown with shRNA (GPR173) in the CHO-GPR173 Cells

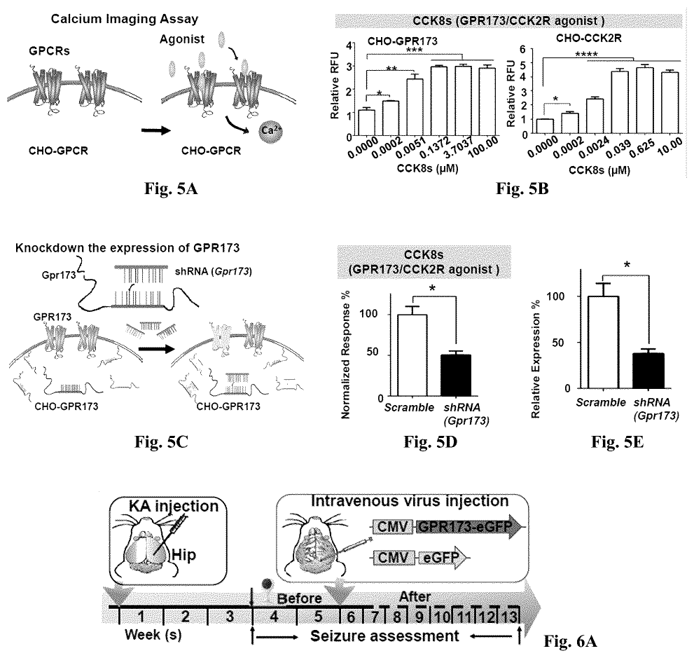

The coding sequence of Hygromycin B is PCR-amplified by Phanta Max Super-Fidelity DNA Polymerase (Vazyme Biotech, http://vazyme.bioon.com.cn/, Nanjing, China) from other plasmids. The purified Hygromycin B fragment and the pLKO.1-puro plasmid (Addgene, Cambridge, Mass, US, https://www.addgene.org) are both digested by BamHI and KpnI restriction enzyme (New England Bio Labs, Mass, USA, https://www.neb.com). The resulting fragments are separated by 1.0% agarose gel electrophoresis and retrieved by DNA retrieve KIT (TIANGEN Biotech, http://www.tiangen.com, Beijing, China). The two BamHI and KpnI fragments are conjoined by T4 DNA Ligase (New England Bio Labs, Mass, USA, https://www.neb.com). The generated plasmid is termed as pLKO.1-hygro. Then, the selected shRNA sequence is designed and cloned into pLKO.1-hygro plasmid by AgeI/EcoRI sites. The most effective shRNA (GPR173) sequence is determined as follows: ACGTGGGCACCTACAAGTTTA, the scramble sequence (scramble) is as follows: CCTAAGGTTAAGTCGCCCTCG. A GPR173 knockdown cell line is generated using the method described above. After lentivirus infection of CHO-GPR173 cells, these cells are screened by 400 μg/ml Hygromycin B (Thermo Fisher, Waltham, MA, US, https://www.thermofisher.cn) for at least one week (FIGS. 5C-5D).

Quantitative Real-Time PCR (qRT-PCR)

Primer synthesis is carried out by GENEray biotechnology, Shanghai, China, and the sequences are shown as follows:

gpr 173-
TCTGGTCACCCTACATCGTG/CAGTAGGGTTCTCTGGGAGC,
actb-
CCTCTATGCCAACACAGTGC/CCTGCTTGCTGATCCACATC.

After extracting the total RNA by RNAsimple Total RNA Kit (TIANGEN Biotech, http://www.tiangen.com, Beijing, China), 1.6 μg RNA, and FastKing RT Kit (TIANGEN Biotech, http://www.tiangen.com, Beijing, China) are used to generate cDNA according to the manufacturer's instructions. Then the primers are amplified with SYBR Green Mix (TIANGEN Biotech, http://www.tiangen.com, Beijing, China) through the 2^{-ΔCt} method. Actb is an endogenous control gene for gpr173. All the procedures are repeated three times in triplicate.

### Calcium Imaging Assay

All measurements of calcium imaging are performed by EnVision 2104 Multilabel Reader (Perkin Elmer, Waltham, Massachusetts, US, https://www.perkinelmer.com, excitation: 485 nm and emission: 535 nm). 6×10⁴ cells of each well growing overnight on 96-well optical-bottom plates with cover glass base (Corning, New York, US, https://www.corning.com), are measured by Fluo-8 No Wash Calcium Assay Kit (AAT-bioquest, California, US, https://www.aatbio.com), the most effective dye to detect intracellular free Ca²⁺. Cells are cultured at 37° C., 5% CO₂ in DMEM/F-12 medium (Thermo Fisher, Waltham, MA, US, https://www.thermofisher.cn) containing 10% FBS (Natocor-Industria Biológica) and washed with DMEM/F-12 medium once after overnight incubation.

### Live-Cell Fluorescent Ligand Binding Assay

For live-cell fluorescent ligand binding assay, 8×10⁴ cells are grown at 37° C., 5% CO₂ for 24 h on 8-well chambered coverglass (Thermo Fisher, Waltham, MA, US, https://www.thermofisher.cn). Cells are washed three times with HHBS solution (HanK's solution supplemented with 20 mM HEPEs, pH=7.4) before equilibration with 360 μL HHBS solution for 15 min. Images are obtained by Zeiss 710 NLO Confocal Microscope with 40× objective. For a competitive live-cell fluorescent ligand binding assay, the cells are incubated with the agonist for 5-15 minutes before adding the fluorescent ligand. Images within each experiment are collected using identical laser-power, offset, and gain.

### Behaviors

### Intrahippocampal Kainic Acid (KA) Injection-Induced Chronic Epilepsy Model

### Surgery

C57BL/6J male mice (6˜7 weeks old) are anaesthetized with pentobarbital (100 mg/kg), a craniotomy is performed perpendicularly to the CAT region of the hippocampus (−2.06 mm posterior to the bregma, −1.80 mm to the midline, and −1.60 mm to the dura), and 650 nL KA (0.3 mg/mL) is injected at a speed of 30 nL/min.

Upon recovering from the anesthesia, the mice are monitored for at least an hour for any signs of acute epileptic seizures before being placed back into their cages for routine monitoring. For the mice only injected with KA (FIG. S16), they are monitored for 16 weeks after the KA injection.

### Observation and Assessment of Epilepsy by Mice Movement Recognition System (MMRS)

The mice are housed separately under a standard 12-h light/dark cycle and stable temperature (23-25° C.) with free access to food and water. Each KA-injected mouse is monitored and recorded 24 h per day using a 360-smart camera for a minimum of three weeks after surgery.

Software is developed based on deep learning architecture (Pytorch and Python development environment) to accomplish automatic epileptic ictal recognition. The MMRS is composed of three main parts (FIG. 6, Video Extraction, Frame Tabbing, movement Prediction, and seizure confirm by manually check). First, the histogram equalization is proposed to enhance the contrast of video frames by equalizing pixels of different grayscale in grey frames. The difference between three frames (previous frame, current frame, and last frame) is computed, and then a threshold k is used to detect the noise in the difference. If the difference in the same pixel of the two frames is less than k, then the difference is treated as noise and decreased to 0 (greyscale value). Otherwise, the difference is sharpened to 255 (greyscale value). A summation of each pixel value for the difference is used to decide whether the animal moved between these three frames. If the animal did not, a video interval between these frames is deleted. As a result, some parts of the video are deleted when the animal is sleeping or inactive.

In the next step, histogram equalization is used to enhance the contrast of video frames. Continuous 64 RGB frames stream in 1 min test video are extracted. Based on the moving mice databank model trained by fine-tuning our dataset under 3d ResNext-101, which video clips show movement occurrences are predicted. Video clips containing suspected seizure-like movement are blindly manually confirmed after the original videos are screened out by a computer program.

The seizures of the mice are evaluated in a blinded manner; the examiners of the video are blind to the history of the mice. The seizure intensity are categorized into five stages according to the Racine scale: 1) mouth and facial movements; 2) head nodding; 3) forelimb clonus; 4) rearing; 5) rearing and falling (Luttjohann, A., et al., Physiol. Behav., Vol. 98, pp. 579-586, 2009). Only stages 3-5 are considered in our analysis.

### AAV-PHP.eB Intravenous Injection in Epileptic Mice

After the C57bL/6J male mice (9-10 weeks old) show a stable occurrence of spontaneous recurrent seizures, epileptic mice are randomly divided into two groups (experimental and control). The daily seizure occurrence is recorded for 2 weeks as the pre-treatment baseline for each mouse (FIG. 6).

The epileptic mice are then anaesthetized with pentobarbital sodium (80 mg/kg) and atropine (0.05 mg/kg, Sigma-Aldrich, St. Louis, MO, USA; https://www.sigmaaldrich.cn/). The skull near the lambda is opened to expose the transverse venous sinus for virus injection. The experimental group mice receive an injection of rAAV-CMV-GPR173-eGFP-PHP.eB-WPREs (CMV-GPR173-eGFP, 5.01 E+12 vg/mL), while the control group rAAV-CMV-eGFP-PHP.eB-WPREs (CMV-eGFP, 5.32 E+12 vg/mL). The PHP.eB serotype virus, which readily crosses the BBB for brain transduction, is produced using standard production methods. A glass pipette tip is used (at an angle of ˜30-45°) to slowly touch the surface of the transverse venous sinus. After the glass pipette tip penetrates the vessel, the injection of a total of 50 μL of the virus at a speed of 1,000 nL/min is performed.

The mice are monitored for seizure occurrences for another 8 weeks after the CMV-GPR173-eGFP or CMV-eGFP intravenous injection.

### Spontaneous Locomotion Detection in Mice

Animal movements are analyzed with the same self-developed program. The active time (movement, dining, grooming, exploring, etc.) is calculated for each mouse every 24 h.

### Quantifications, Statistics, and Reproducibility

Quantifications are obtained using a minimum of three independent biological replicates. Data collection and analysis are not performed in a blinded manner if not specified, but different research groups perform the quantifications. Group data are presented as the mean±SEM. SPSS 25.0 (IBM) and Excel are used to perform statistical analyses, including Student's unpaired t-test, Student's paired t-test, one-way ANOVA with Tukey's post hoc test, and two-way RM ANOVA with Tukey's post hoc test. Asterisk or sharp symbols denote significant differences in all figures (*P<0.05, **P<0.01, ***P<0.001, ****P<0.0001, and ####P<0.0001). N.S. indicates that a difference is not significant. OriginPro 8.5 (OriginLab Corporation), Inkscape 0.92.3 (Inkscape), and GraphPad Prism 8 (GraphPad Software) are used to perform the graphing. The reference brain map with anatomical abbreviations is accessible on the Allen Brain Atlas website (https://portal.brain-map.org/).

TABLE 1

| | | |
|---|---|---|
| REAGENT | | |
| RESOURCE | SOURCE | IDENTIFIER |

Antibodies

| | | |
|---|---|---|
| Anti-GAD67 | Sigma-aldrich (St. Louis, MO, US; | Mab5406 |
| | https://www.sigmaaldrich.cn/) | |
| Anti-ChR2 | ARP (Promega Corporation, | 03-651180 |
| | Madison, Wisconsin, | |
| | https://www.promega.com.cn) | |
| Anti-CCK | Sigma-aldrich | C2581 |
| Anti-PV | Abcam (Cambridge, UK; | Ab11427 |
| | https://www.abcam.com) | |
| Anti-GABAAR | Santa Cruz (Santa Cruz, California, | Sc-376282 |
| | US, https://www.scbt.com) | |
| Anti-GABAAR | Abcam (Cambridge, UK; | Ab7291 |
| | https://www.abcam.com) | |
| Anti-mCherry | ThermoFisher (Waltham, MA, US, | M11217 |
| | https://www.thermofisher.cn) | |
| Anti-GFAP | Santa Cruz (Santa Cruz, California, | Sc-33673 |
| | US, https://www.scbt.com) | |
| Anti-Camkll | Abcam (Cambridge, UK; | Ab5683 |
| | https://www.abcam.com) | |
| | (Cambridge, UK; | |
| | https://www.abcam.com) | |
| Anti-Camkll | Abcam (Cambridge, UK; | Ab325403 |
| | https://www.abcam.com) | |
| Anti-Flag | Cell Signaling Technology (Boston, | CST-8146 |
| | US, https://www.cellsignal.com) | |
| Anti-HA | Cell Signaling Technology | CST-3724 |
| anti-Synaptophysin 1 | synaptic systems (SYSY) | 101 004 |
| Anti-GPR173 | ThermoFisher (Waltham, MA, US, | PA5-50976 |
| | https://www.thermofisher.cn) | |
| Anti-GPR85 | ThermoFisher (Waltham, MA, US, | PA5-33776 |
| | https://www.thermofisher.cn) | |
| Anti-CCKIR | Novus (St. Louis, Missouri, | AF2680 |
| | https://www.novusbio.com) | |
| Anti-CCK2R | Affinity Bioscience (Bei Jing, | DF4861 |
| | China, http://www.affbiotech.cn) | |
| Anti-GPR15 | Affinity Bioscience (Bei Jing, | DF4965 |
| | China, http://www.affbiotech.cn) | |
| Anti-BRS3 | Santa Cruz (Santa Cruz, California, | sc-271712 |
| | US, https://www.scbt.com) | |
| Anti-GPR17 | Santa Cruz (Santa Cruz, California, | sc-514723 |
| | US, https://www.scbt.com) | |
| Anti-GPR135 | Affinity Bioscience (Bei Jing, | DF4948 |
| | China, http://www.affbiotech.cn) | |
| Anti-GPR132 | Santa Cruz (Santa Cruz, California, | sc-137112 |
| | US, https://www.scbt.com) | |
| Anti-TAAR9 | Novus (St. Louis, Missouri, | NLS1969 |
| | https://www.novusbio.com) | |
| Anti-GPR19 | Affinity Bioscience (Bei Jing, | DF2729 |
| | China, http://www.affbiotech.cn) | |
| Anti-GPR34 | Affinity Bioscience (Bei Jing, | DF4972 |
| | China, http://www.affbiotech.cn) | |
| Anti-GPR83 | Affinity Bioscience (Bei Jing, | DF2768 |
| | China, http://www.affbiotech.cn) | |
| Anti-GPR45 | Absin (Beijing, China, | abs112926 |
| | https://www.absin.cn/) | |
| Anti-TAAR5 | Novus (St. Louis, Missouri, | NBP1-68902 |
| | https://www.novusbio.com) | |
| Anti-GPR20 | Affinity Bioscience (Bei Jing, | DF2730 |
| | China, http://www.affbiotech.cn) | |
| Goat anti Gp 405 | Abcam (Cambridge, UK; | ab175678 |
| | https://www.abcam.com) | |
| Goat anti Gp 488 | Abcam (Cambridge, UK; | ab150185 |
| | https://www.abcam.com) | |
| Goat anti Mouse 488 | Abcam (Cambridge, UK; | ab150113 |
| | https://www.abcam.com) | |
| Goat anti Rabbit 647 | Abcam (Cambridge, UK; | ab150079 |
| | https://www.abcam.com) | |
| Goat anti Mouse 568 | Abcam (Cambridge, UK; | ab175473 |
| | https://www.abcam.com) | |
| Goat anti Rabbit 488 | Abcam (Cambridge, UK; | ab150077 |
| | https://www.abcam.com) | |
| Goat-anti-mouse-594 | ThermoFisher (Waltham, MA, US, | 35511 |
| | https://www.thermofisher.cn)Fisher | |
| Goat-anti-rat-488 | ThermoFisher (Waltham, MA, US, | A11006 |
| | https://www.thermofisher.cn)Fisher | |
| Donkey-anti-mouse-488 | Jackson Immuno Research | 715-545-150 |
| | (Pennsylvania, US, | |
| | https://www.jacksonimmuno.com) | |
| Donkey-anti-mouse-594 | Jackson Immuno Research | 715-585-150 |
| | (Pennsylvania, US, | |
| | https://www.jacksonimmuno.com) | |
| Donkey-anti-mouse-647 | Jackson Immuno Research | 715-605-150 |
| | (Pennsylvania, US, | |
| | https://www.jacksonimmuno.com) | |
| Donkey-anti-rabbit-488 | Jackson Immuno Research | 711-545-152 |
| | (Pennsylvania, US, | |
| | https://www.jacksonimmuno.com) | |
| Donkey-anti-rabbit-594 | Jackson Immuno Research | 711-585-152 |
| Goat-anti-rabbit-750 | ThermoFisher (Waltham, MA, US, | A-21039 |
| | https://www.thermofisher.cn)Fisher | |
| Goat-anti-mouse-750 | ThermoFisher (Waltham, MA, US, | A-21037 |
| | https://www.thermofisher.cn)Fisher | |
| Goat-anti-rat-647 | Abcam (Cambridge, UK; | ab150159 |
| | https://www.abcam.com) | |
| Donkey-anti-rabbit-647 | Jackson Immuno Research | 711-605-152 |

Chemicals, peptides, and recombinant proteins

| | | |
|---|---|---|
| A-71623 | Tocris (Bristol, UK, | 2411 |
| | https://www.tocris.com/) | |
| CCK4 | BankPeptide (He Fei, China, | N/A |
| | http://www.bankpeptide.com) | |
| CCK8s | BankPeptide (He Fei, China, | N/A |
| | http://www.bankpeptide.com) | |
| PNX | BankPeptide (He Fei, China, | N/A |
| | http://www.bankpeptide.com) | |
| CCK8 | BankPeptide (He Fei, China, | N/A |
| | http://www.bankpeptide.com) | |
| F-CCK8 | BankPeptide (He Fei, China, | N/A |
| | http://www.bankpeptide.com) | |
| TAMRA-CCK4 | BankPeptide (He Fei, China, | N/A |
| | http://www.bankpeptide.com) | |
| HA2A | BankPeptide (He Fei, China, | N/A |
| | http://www.bankpeptide.com) | |
| HA2A8s | Ke Biochem (Shang Hai, China, | N/A |
| | http://en.kebiochem.com) | |

Critical commercial assays

| | | |
|---|---|---|
| Calcium Imaging assay | AAT Bioquest (Beijing, China, | 36315 |
| kit | https://www.aatbio.com) | |

Experimental models: Cell lines

| | | |
|---|---|---|
| CHO-CCKIR (CHO | The invention | N/A |
| cell line stably | | |
| expressing CCK1R) | | |
| CHO-CCK2R (CHO | The invention | N/A |
| cell line stably | | |
| expressing CCK2R) | | |
| CHO-GPR173 (CHO | The invention | N/A |
| cell line stably | | |
| expressing GPR173) | | |
| CHO | Cell Bank of Guangzhou Institutes | N/A |
| | of biomedicine and health, Chinese | |
| | academy of sciences, | |
| | (http://english.gzb.cas.cn/) | |
| HEK293T | ATCC (Rockville, MD, US, | CRL-3216 |
| | https://www.atcc.org) | |

Experimental models: Mice

| | | |
|---|---|---|
| Vgat-Cre: Vgat-ires- | The Jackson Laboratory (Bar | JAX: 016962 |
| Cre^{Slc32a1tm2(Cre)Lowl}/J | Harbor, Maine, US, | |
| | https://www.jax.org) | |
| CCK-cKO: mCCK- | Cyagen (Guangzhou, China, | N/A |
| Conditional-KO | https://www.cyagen.com) | |
| CCK-Cre: CCK-ires- | The Jackson Laboratory (Bar | JAX: 019021 |
| Cre (Cck^{tm1.1(Cre)Zjh}/J) | Harbor, Maine, US, | |
| | https://www.jax.org) | |
| PV-Cre: PV-ires-Cre | The Jackson Laboratory (Bar | JAX: 017320 |
| (Pval^{btm1(cre)Arbr}/J) | Harbor, Maine, US, | |
| | https://www.jax.org) | |
| CCK1R/2R-KO: CCK- | The Jackson Laboratory (Bar | JAX: 006365 |
| AR/BR-KO (Cckar^{tm1Kpn} | Harbor, Maine, US, | |
| Cckbr^{tm1Kpn}/J) | https://www.jax.org) | |
| CCK-KO: CCK-CreER | The Jackson Laboratory (Bar | JAX: 012710 |
| (Cck^{tm2.1(Cre/ERT2)Zjh}/J) | Harbor, Maine, US, | |
| | https://www.jax.org) | |

Oligonucleotides

| | | |
|---|---|---|
| PCR primers | The invention | See methods |
| Primer for Vgat-Cre | BGI (Shenzhen, China, | CAGGGCGATGTGGAATAG |
| | https://www.bgi.com) | AAA |
| | | CTTCGTCATCGGCGGCAT |
| | | CTG |
| | | CCA |
| | | AAAGACGGCAATATGGT |
| Primer for CCK-cKO | BGI (Shenzhen, China, | GAAGGATGCCAGGAAAG |
| | https://www.bgi.com) | GTGGTAGC |
| | | CACATGGAAACACAGCCT |
| | | ATCGTTCC |
| Primer for CCK-Cre | BGI (Shenzhen, China, | GAGGGGTCGTATGTGTGG |
| | | TT |
| | | GGGAGGCAGATAGGATCA |
| | | CA |
| | https://www.bgi.com) | TGGTTTGTCCAAACTCATC |
| | | AA |

### Example 1

### CCK-GABA Neurons Potentiate the Inhibition in the Auditory Cortex

An in vivo recording animal model is adopted for this example. It is believed that activation of the GABAergic neurons induces inhibitory outputs to the surrounding neurons in the neocortex. The inventors assume that prior activation of GABAergic neurons would suppress neuronal responses to the forthcoming auditory stimulus (AS) in the auditory cortex (AC).

Whether the HFLS of GABAergic neurons could potentiate their inhibition onto neuronal responses to the forthcoming AS in the AC is examined. Next, whether CCK is involved in potentiating their inhibitory outputs towards their targeted neurons is determined.

A CCK conditional knockout mice (mCCK-cKO) are generated using the CRISPR/Cas9 system. To purposefully knock out CCK in the GABAergic neurons (CCK^{-/-}-GABA), Vgat-Cre and mCCK-cKO mice are crossed. A double loxP-flanked (DIO) Cre-dependent AAV vector expressing channelrhodopsin-2 variant (ChETA) and enhanced yellow fluorescent proteins (eYFP, AAV-EF1a-DIO-ChETA-eYFP, 8E+12 VG/mL, VG: vector genome) is injected in the AC of Vgat-Cre and Vgat-Cre-CCK-cKO mice infecting GABAergic neurons specifically. Four AC locations are injected with 300 nL of the AAV vector at a 30 nL/min speed for each site and 0.65 mm depth under dura. Most eYFP⁺ neurons co-localize with glutamic acid decarboxylase 67 (GAD67), an inhibitory neuronal marker. It is found that CCK is void in GABAergic neurons of the Vgat-Cre-CCK-cKO mouse but intact in the Vgat-Cre mouse, and most of the labelled neurons are GAD67 positive, including PV subtypes in Vgat-Cre-CCK-cKO.

In the in vivo setup for the anaesthetized mouse, an optic fiber is inserted into the AC to activate the infected GABAergic neurons and a high-impedance glass electrode array that recorded multiunit responses and local field potentials (LFP) is inserted. The normalized sound intensity and laser power are selected after measuring the input-output curve for stimulus intensities for each mouse. The kinetics of optogenetics opsin is examined by applying laser pulse trains of different frequencies (1 to 120 Hz) for both groups. It is found that the laser activation of the GABAergic neurons causes a lasting inhibition in the spontaneous firing of about 200 ms. The AS of 100 ms duration is delivered at 50 ms after the laser pulse delivery. The window of AS presentation is adopted to exclude the possible effects of the post-inhibitory rebound excitation, recurrent inhibition, and oscillatory activities in the examination of the inhibitory effect by activating GABAergic neurons.

Laser-activated GABAergic neurons inhibit neuronal response to the forthcoming AS in Vgat-Cre and Vgat-Cre-CCK-cKO mice. The following HFLS protocol is adopted: HFLS, 5-pulse bursts in 40 Hz, 10 s of inter-burst-interval, 200 pulses in total. HFLS of GABAergic neurons potentiate the inhibition of laser-activated GABAergic neurons on the neuronal response to the forthcoming AS in Vgat-Cre mice (group data of firing rate: the firing rate reduced from 113.08±36.92 Hz (before the HFLS) to 63.86±17.82 Hz (after the HFLS), n=16 units, N=11 mice, paired two-tailed t-test, ***P<0.001). No potentiation of the inhibition of the Vgat-Cre-CCK-cKO mice is found (group data of firing rate: 116.63±17.79 Hz before the HFLS vs. 120.97±16.98 Hz after the HFLS, n=14 units, N=11 mice; paired two-tailed t-test; N.S., not significant). The spontaneous firing rate of the two groups remain the same before and after HFLS. To exclude the possible laser-induced heat and photo-electrical effects, the Vgat-Cre mice injected with DIO-eYFP are recruited (FIGS. S5A-S5B) and no potentiated inhibition after HFLS is observed (group data of firing rate: 141.90±21.98 before the HFLS vs. 134.09±21.17 Hz after the HFLS, n=15 units, N=10 mice; paired two-tailed t-test; N.S.).

In addition, the HFLS induces a distinct reduction in amplitude of LFP to AS in Vgat-Cre mice, but not in Vgat-Cre-CCK-cKO mice (Vgat-Cre HFLS: 52.75±6.84% vs. Vgat-Cre-CCK-cKO HFLS: 103.13±2.34%, two-way RM ANOVA with Tukey's post hoc test, ####P<0.0001).

In summary, HFLS of local GABAergic neurons potentiates the inhibition of GABAergic neurons onto the neuronal response to the forthcoming AS in the Vgat-Cre mouse, but not in the Vgat-Cre-CCK-cKO mouse. The potentiated inhibition is long-lasting during the observation period of 60 min.

### Example 2

### HFLS of CCK-GABA Neurons, but not PV Neurons, Potentiates their Inhibitory Postsynaptic Current (IPSC) in Glutamatergic Neurons

The above example shows that the HFLS of GABAergic neurons in the Vgat-Cre mouse, but not in the Vgat-Cre-CCK-cKO mouse induces potentiated inhibition of neuronal activities in the AC. In the present example, the patch-clamp recording is used on pyramidal neurons to examine whether the potentiation of inhibition of GABAergic inputs is CCK-GABA neurons specific. CCK-GABA neurons are targeted, and PV neurons are recruited as the control since PV neurons represent the most significant proportion of GABAergic neurons in the cortex (Gonchar, Y., Front. Neuroanat., Vol. 1, pp. 1-11, 2008; Whissell, P. D., et al., Front. Neuroanat., Vol. 9, p. 124, 2015).

To virally target PV or CCK-GABA neurons, the Cre-dependent AAV virus is injected into the AC of the PV-Cre or CCK-Cre mouse. The inventors design a Channelrhodopsin-2 (ChR2) and fluorescent protein reporter (mCherry) expressing AAV, containing a woodchuck hepatitis virus post-transcriptional regulatory element (WPRE) under the control of the inhibitory neuron-specific Distal-less homeobox (mDlx) gene enhancer sequence (rAAV9-mDlx-DIO-ChR2-mCherry-WPRE-pA, hereinafter referred to as mDlx-DIO-ChR2-mCherry, FIG. 1A). mDlx enhancer restricts specific and robust expression to interneurons across vertebrate species (Dimidschstein, J., et al., Nat. Neurosci., Vol. 19, pp. 1743-1749, 2016). It is found that our virus labels mostly CCK-GABA neurons in CCK-Cre mice and PV neurons in PV-Cre mice (96.3% mCherry labeled neurons are GAD67⁺, 95.4%±1.6% CCK⁺, and 96.7±1.2% ChR2⁺ on CCK-Cre; 97.5%±1.0% mCherry labeled neurons are PV⁺, and 98.2±1.0% ChR2⁺ on PV-Cre).

The brain slices of CCK-Cre mice, or PV-Cre mice are used for in vitro patch-clamp recording (FIG. 1B). A pyramidal neuron (FIG. 1B for the morphology visualized by Biotin-488 and physiological properties examined by the response to injected step currents) in the virus-injected area of the AC is selectively patched. The IPSC evoked by the laser stimulation (0.15 Hz, 5 mins as baseline) of nearby inhibitory neurons is directly recorded. A robust potentiation of IPSC in the CCK-Cre mouse is found after the HFLS while no potentiation of IPSC is found in the PV-Cre mouse (FIG. 1C, single traces, and averaged traces of IPSC responses to the laser stimulation at 0.15 Hz, before and after the HFLS during 0-5 min, 5-15 min, and 15-25 min). Time course of normalized IPSC before (for 5 min) and after the HFLS (for 25 min) shows no significant potentiation of IPSC in the PV-Cre mice (baseline vs. 20-25 min after HFLS: 100.00±2.43% vs. 103.67±7.00%, n=7 neurons, two-way ANOVA with Tukey's post hoc test, P 0.26, N.S.), while the HFLS of CCK-GABA neurons induces a significant potentiation of IPSC in the CCK-Cre mice (baseline vs. 20-25 min after HFLS: 101.56±5.73 vs. 183.70±6.25%, n=9 neurons, one-way ANOVA with Tukey's post hoc test, ****P<0.0001. The averaged IPSC amplitudes of baseline among two group neurons are similar (CCK-Cre vs. PV-Cre: 92.16±12.43 vs. 102.39±19.85 pA, one-way ANOVA with Tukey's post hoc test, N.S.).

In summary, HFLS of CCK-GABA neurons induces a significant potentiation of IPSC during the recording period, or iLTP (HFLS of CCK-GABA vs. HFLS of PV: 103.67±7.00% vs. 183.70±6.25%, two-way RM ANOVA with Tukey's post hoc test, ####P<0.0001).

### Example 3

### Application of CCK8s and LFLS of CCK-GABA Neurons Induce iLTP in Glutamatergic Neurons

Next, the inventor hypothesizes that the HFLS of the CCK-GABA neurons triggers the co-release of GABA and CCK that induced iLTP on their targeted neurons. At the same time, LFLS of the same number of pulses induces no CCK release and thus no iLTP. If true, direct application of CCK could generate iLTP, even followed by the LFLS of CCK-GABA neurons.

To test the hypothesis, the same in vitro patch-clamp recording is adopted in pyramidal neurons in the AC where CCK-GABA neurons are selectively transfected with the AAV virus (mDlx-DIO-ChR2-mCherry; same as FIG. 1A). CCK8s or artificial cerebrospinal fluid (aCSF) is applied followed by the LFLS (FIG. 2A). A large iLTP is observed in the CCK8s+1 Hz group and no iLTP is found in the aCSF+1 Hz group. (FIG. 2B, single and averaged traces of IPSC; normalized IPSC amplitudes: baseline vs. 25-30 min after CCK8s+1 Hz, 100.00±2.53 vs. 161.84±4.38%, n=8 neurons; baseline vs. 25-30 min after aCSF+1 Hz, 100.10±1.96 vs. 92.05±6.58%, n=7 neurons, two-way ANOVA with Tukey's post hoc test, ****P<0.0001; N.S.). The averaged IPSC amplitudes of baseline among two group neurons are similar (CCK8s+1 Hz vs. aCSF+1 Hz: 87.16±18.78 vs. 128.90±22.73 pA, one-way ANOVA with Tukey's post hoc test, N.S.).

Therefore, the application of CCK8s that followed LFLS of the CCK-GABA neurons induces iLTP compared to no LTP in the aCSF control (25-30 min after aCSF+1 Hz vs 25-30 min after CCK8s+LFLS between groups: two-way RM ANOVA with Tukey's post hoc test, ####P<0.0001).

### Example 4

### Known CCK Receptors do not Mediate the CCK Signaling of CCK-GABA Neurons

The next is to determine which CCK receptor mediates iLTP of CCK-GABA neurons. There are two known CCK receptors in the brain: CCK1R and CCK2R. CCK2R is widely distributed throughout the CNS, whereas CCK1R is only found in certain regions such as the hypothalamus, the nucleus accumbens, and the hippocampus (Honda, T., et al., Mol. Cell. Neurosci., Vol. 4, pp. 143-154, 1993). It is believed that CCK2R mediates the entorhinal-cortical CCK's action for cortical LTP and the encoding of associative memory (Li, X., et al., Cell Res., Vol. 24, pp. 307-330, 2014; Chen, X., et al., Proc. Natl. Acad. Sci., Vol. 116, pp. 6397-6406, 2019).

CCK1R/2R-KO and CCK-KO mice are adopted to examine what CCK receptor mediates the CCK signaling of CCK-GABA neurons for iLTP in the brain slice experiment before examining specific CCK2R-KO or CCK1R-KO mice.

Two viruses (FIGS. 3A-3C: mDlx-DIO-ChR2-mCherry, 2.09 E+12 vg/mL; and mDlx-Cre-helper rAAV9, 1.42 E+12 vg/mL) are combined, which both contain mDlx, and restrict the virus expression in the interneurons. Total 1,200 nL mixture of viruses (one carries ChR2 and the other carries Cre) are injected into the AC of CCK1R/2R-KO or CCK-KO mice.

Immunohistochemical experiments are conducted to examine whether the combination of two viruses expresses in the target GABAergic neurons effectively and precisely. Confocal images show that almost all infected neurons are GABAergic neurons in both mouse lines (one-way ANOVA with Tukey's post hoc test, N.S.). Adopting the same protocol of combined laser and AS in Example 1, the suppression of laser activation of GABAergic neurons upon the neuronal responses to forthcoming AS in the AC is examined. The results show that, HFLS of GABAergic neurons induces no reduction in amplitude of LFP to the AS in CCK-KO mice. To our surprise, HFLS of GABAergic neurons induces a distinct decrease in the amplitude of LFP response to the AS in the CCK1R/2R-KO mice (CCK1R/2R-KO: 63.56±5.09% vs. CCK-KO: 100.17±4.20%, two-way RM ANOVA with Tukey's post hoc test, ####P<0.0001), indicating a novel CCK receptor other than CCK1R and CCK2R is involved in mediating the signaling of CCK from CCK-GABA neurons.

The experiment is reconfirmed by using in vitro patch-clamp recording. Pyramidal neurons are selected as the recording targets (FIG. 3B, the preparation for the recording and the response to current injection). HFLS of GABAergic neurons induces iLTP in the recorded pyramidal neuron in CCK1R/2R-KO mice, but no iLTP in CCK-KO mice (FIG. 3C for single and averaged traces, CCK1R/2R-KO mice, baseline vs. 20-25 min after the HFLS: 100.36±2.91 vs. 150.34±5.32%, n=9 neurons, two-way ANOVA with Tukey's post hoc test, ****P<0.0001; CCK-KO mice, baseline vs. 20-25 min after the HFLS: 99.93±2.22 vs. 97.71±2.98%, n=9 neurons, two-way ANOVA with Tukey's post hoc test, P 0.82, N.S.). In summary, the above results indicate that the CCK-enabled iLTP is mediated neither by CCK1R nor CCK2R.

### Example 5

### Proteomics Screen Out and Cell-Line Assay Confirmation of the Potential Novel CCK Receptor

The inventors suspect that the potential new CCK receptor, which mediates the CCK8s signaling of CCK-GABA neurons, is a G-protein coupled receptor (GPCR), similar to CCK1R and CCK2R. Using the surface accessibility of extra-membrane proteins (Petersen, B., et al., BMC Struct. Biol., Vol. 9, p. 51, 2009), all other GPCRs are compared with CCK1R and CCK2R based on hamming distance (http://www.clustal.org). Several GPCRs are found as potential candidates of CCK receptors (FIG. 4A).

To further evaluate which GPCRs listed in FIG. 4A are the candidates of the new CCK receptor, the Flag-tagged GPCRs cell lines are established accordingly, and CCK-binding assays are developed. HA-labeled CCK8s are incubated with HEK293T cells transfected with N-terminal Flag-tagged GPCRs for the membrane surface labelling assay. HA2A8s (HA-ε-Ahx-ε-Ahx-CCK8s) is composed of one HA tag, two ε-Ahx (6-aminocaproic acid) linkers, and one CCK8s sequence (an active form of CCK in the brain); HA2A (HA-ε-Ahx-ε-Ahx) is the negative control (FIG. 4B). The results in FIG. 4B show that HA2A8s can bind to Flag-CCK2R, Flag-CCK1R, and Flag-GPR173, but not other candidates. Besides, the specific binding character of Flag-GPR173 with CCK8s is demonstrated by competitive membrane surface labelling assay of Flag-GPR173, incubated together with CCK8s and HA2A8s (n=33 to 40 cells, one-way ANOVA with Tukey's post hoc test, ****P<0.0001, N.S.). Confocal immunostaining images for ACGFP-CCK1R with HA2A (negative control) or HA2A8s validate the effectiveness of CCK-binding assays by CCK-1R cells and HA2A8s.

According to the CCK-binding assay and bioinformatics prediction, the inventors surprisingly found that an orphan receptor, GPR173 has relatively higher similarity scores of 0.450643 against CCK1R and 0.398852 against CCK2R (FIG. 4A), and the prominent binding affinity of Flag-GPR173 with CCK8s (FIG. 4B).

The above findings lead us to the hypothesis that the sequences of CCK receptor candidates are closely correlated with CCK1R/2R sequences. Clustal (online software, http://www.clustal.org) is used to extract the information of the neighboring joining tree (Saitou, N., et al., Mol. Biol. Evol., Vol. 4, pp. 406-25, 1987) of the 79 orphan receptors. The percentage identity correlation score defines the quantitative correlation estimation between two sets of sequences (Raghava, G., et al., BMC Bioinformatics, Vol. 7, p. 415, 2006). According to the percentage identity correlation score among 79 orphan receptors and CCK1R/2R, CCK1R, CCK2R, and 7 orphan receptors are selected from 79 orphan receptors for anatomical examination (FIG. 4C).

### Example 6

### Anatomical Confirmation for the Potential CCK Receptors

The inventors hypothesize that if the novel CCK receptor mediates CCK signaling of CCK-GABA neurons on their postsynaptic neurons, the novel receptor should localize near the CCK-GABA synapses. To test the above hypothesis, 600 nL of mDlx-DIO-ChR2-mCherry (2.09 E+12 vg/mL) is injected in the AC of the CCK-Cre mouse. The possible co-localization between GPCRs and CCK-GABA synapses is examined by adopting triple-labelling of mCherry, synaptophysin, and the 7 orphan receptors with the highest correlation scores (FIG. 4D). Confocal images of Flag-GPR173 transfected HEK293T co-labelled with anti-GPR173, anti-Flag and DAPI are taken for quantitative analysis of colocalization of Flag and GPR173. The results show that the GPR173 antibody is validated on the Flag-GPR173 transfected HEK293T cells.

It is found that GPR173 has the highest co-localization density with CCK-GABA synapses (FIG. 4D) compared with other 15 GPCRs including GPR85, CCK1R, CCK2R, GPR15, BRS3, GPR17, GPR135, GPR132, TAAR9, GPR19, GPR34, GPR83, GPR45, TAAR5, and GPR20 (3 independent experiments, GPR173 vs GPR85, the highest among others, 948.90±35.36 vs 53.46±17.68 numbers/mm², one-way ANOVA with Tukey's post hoc test, ***P<0.0001), indicating that GPR173 is the most likely GPCR mediating CCK-GABA signaling.

An increased number of activated GABA_{A}R is associated with lasting potentiation of inhibition (Otis, T. S., et al., Proc. Natl. Acad. Sci., Vol. 91, pp. 7698-7702, 1994). Further anatomical examinations reveal that GPR173 co-localizes with GABA_{A}R, presenting within CCK-GABA neurons' synapses. FIG. 4E shows that the CCK-GABA terminal and GPR173 are co-localized within but slightly segregated from each other with a possible implication of between the pre- and post-synaptic cleft, which is in accordance with CCK-GABA terminal and GABA_{A}R. GPR173 and GABA_{A}R are also co-localized within 0.1 μm (FIG. 4F). The data of super-resolution images (FIG. 4G) are analyzed using Pearson's correlation coefficient (PCC) and Manders' overlap coefficient (MOC) (Aaron, J. S., et al., J. Cell Sci., Vol. 131, pp. 1-10, 2018), which are common metrics to measure the predictability of co-localization, to determine the co-occurrence and correlation coefficients of GPR173 and CCK-GABA terminals. The numerical values of PCC and MOC show higher than 0.78 across 6 groups (FIG. 4G), indicating the great possibility of GPR173 located in the CCK-GABA synapse and near the GABA_{A}R.

The co-staining of GPR173 with Camkll and GAD67⁺ is conducted on CCK-Cre mice to examine the cell type where GPR173 are located. Interestingly, 91.27±1.48% of GPR173⁺ cells are Camkll⁺ neurons (n=11 sections from 3 C57BL/6J wild type mice) and 9.82±1.42% are GAD67+(n=10 sections from 3 C57BL/6J wild type mice) in the auditory cortex.

Among the most-closely correlated 7 GPCRs with CCK1R and CCK2R, GPR173 has the highest overlay score for co-localization with the CCK-GABA terminal. Putting together that GPR173 is also closely located with GABA_{A}R, GPR173 is anatomically the most likely novel GPR173 mediating the CCK signaling from CCK-GABA neurons. GPR173 are situated mostly on excitatory neurons, though less than one-tenth on inhibitory neurons.

### Example 7

### Functional Confirmation of GPR173 as a Novel Receptor that Mediates CCK8s Signaling

Both CCK1R and CCK2R could couple with G_{q} protein, by which CCK1/2Rs promoted the release of intracellular calcium in the endoplasmic reticulum under the agonism effect of their corresponding agonists (Berna, M. J., et al., Curr. Top. Med. Chem., Vol. 7, pp. 1211-1231, 2007). CCK8s, as the potent but non-selective agonist of both CCK1R and CCK2R, has a strong agonistic effect on their G_{q}-PLC-IP₃-Ca²⁺ pathway (Yule, D. I., et al., Am. J. Physiol. Liver Physiol., Vol. 265, pp. G999-G1004, 1993). Since the secondary structure of GPR173 is highly homologous with CCK1/2Rs (FIG. 4A), the inventor hypothesized that GPR173 could also couple with G_{q} protein, and some existing CCK1R/2R agonists might act on GPR173. Stable monoclonal GPCR transgenic CHO cell lines, CHO-CCK1R, CHO-CCK2R (Zhang, Y, et al., Med. Chem. Res., Vol. 28, pp. 387-393, 2019), and CHO-GPR173, are constructed.

Calcium imaging assay shows that CCK8s induces the accumulation of intracellular calcium (FIGS. 5A-5B, EC₅₀=3.23±1.94 nM in the CHO-GPR173 cell line, and EC₅₀=1.61±0.31 nM in the CHO-CCK2R cell line; N=3 independent experiments of two cell lines; one-way ANOVA with Tukey's post hoc test, ****P<0.0001). With the application of shRNA, the expression in the CHO-GPR173 cells is knocked down. The suppression of the calcium signal in the shRNA containing lentivirus infected CHO-GPR173 cells further confirms that exogenous GPR173 mediates the CCK8s-induced increased intracellular calcium signal (FIGS. 5C-5E; statistically decreased expression of GPR173 and suppression of intracellular calcium signal in the shRNA group, N=3 independent experiments; one-way ANOVA with Tukey's post hoc test, *P<0.05).

GPR173, also known as SREB3, is high evolutionary conservation and predominantly expressed in the central nervous system (Matsumoto, M., et al., Mol. Brain Res., Vol. 138, pp. 58-69, 2005). GPR173 has been found as an orphan receptor until it is claimed to mediate Phoenixin (PNX) action recently, as they found that the expression level of GPR173 mRNA is modulated by PNX (Treen, A. K., et al., Mol. Endocrinol., Vol. 30, pp. 872-888, 2016). However, the results show no calcium signal induced in the CHO-GPR173 cells after the incubation with PNX, while a strong calcium response after CCK8s attendance (N=3 independent experiments; one-way ANOVA with Tukey's post hoc test, ****P<0.0001, N.S.). To exclude the possibility of endogenous CCK1R and CCK2R interference in CHO-GPR173 cells, RT-PCR is performed. Confocal images indicate the GPR173 expression in the CHO-GPR173 cells but no expression in control CHO cells. The CCK8s, not PNX, can trigger calcium signals in the CHO-GPR173 cells through the G_{q}-PLC-IP₃-Ca²⁺ pathway. The expression of CCK1R and CCK2R on the cell membrane of their corresponding cell lines is verified by living cell fluorescent ligand binding assay with TAMRA-CCK4 and F-CCK8. CCK1R and CCK2R selective agonists are used to rule out the interference of endogenous CCK1R/2R in CHO-GPR173 cells.

The CCK1R-specific agonist, A-71623 (EC₅₀=3.11±1.80 nM, with 1200-fold selectivity over the CCK2R) activates intracellular calcium accumulation in the CHO-CCK1R cell line. It does not activate the CHO-GPR173 cell line even at 100 μM, indicating no endogenous CCK1R in the CHO-GPR173 cell line (one-way ANOVA with Tukey's post hoc test, *P<0.05, **P<0.01, ***P<0.001, N.S., not significant). Similarly, there is no endogenous CCK2R in CHO-GPR173 cells as the CCK2R-selective agonist, CCK4, evokes no response (one-way ANOVA with Tukey's post hoc test, **P<0.01, ***P<0.001, N.S., not significant).

It is also found that several CCK1R/2R antagonists also showed antagonism to GPR173, including Devazepide (CCK1R antagonist, IC₅₀=12.30±3.41 nM), Loxiglumide (CCK1R antagonist, IC₅₀=715.1±99.44 nM), L365,260 (CCK1R/2R antagonist, IC₅₀=16,850±5,148 nM), and YF476 (CCK2R antagonist, IC₅₀=3,100±603.9 nM). It is interesting to note that Devazepide has a high potency to GPR173 with IC₅₀=12.30±3.41 nM.

In summary, the membrane surface labelling assay, the shRNA technique, and the calcium imaging assay provide convincing evidence that GPR173 is a novel CCK receptor distinguished from the known CCK1R and CCK2R.

### Example 8

### iLTP Blocked by GPR173 Antagonist

It can be understood from the above examples that the HFLS of GABAergic neurons induces iLTP through the CCK signaling from CCK-GABA neurons. Neither CCK1R nor CCK2R mediates the iLTP, as iLTP is intact on CCK1R/2R-KO mice. A novel CCK receptor, GPR173, is identified, which mediates the above signaling and Devazepide is determined as a high-affinity antagonist to GPR173, though it also antagonizes CCK1R. The inventors deduce that if GPR173 mediates the CCK signaling of CCK-GABA neurons, Devazepide should be able to block iLTP.

The above deduction is tested by examining whether the application of Devazepide (at 60 nM in bath solution) block the HFLS-induced potentiation of IPSC. Brain slices of CCK-Cre mice after the injection of GABA-specific virus into the AC (mDlx-DIO-ChR2-mCherry; same as FIG. 1A) are used. Pyramidal neurons in the virus-injected area of the AC are selectively patched. It is found that the application of Devazepide entirely blocks the iLTP (FIG. 2A, potentiation of IPSC; 100.18±1.91 vs. 103.24±2.54%, baseline vs. 20-25 min after Devazepide infusion and the HFLS, two-way ANOVA; post hoc with Tukey HSD, ****p<0.0001, n=8). It contrasts the HFLS without the application of Devazepide (HFLS vs. Devazepide+HFLS between groups: 183.70±6.25%, n=9, vs. 103.24±2.54%, n=8, two-way RM ANOVA, post hoc with Tukey HSD, ****P<0.0001).

Although Devazepide antagonizes both CCK1R and GPR173, the blockage of iLTP is through its antagonism to GPR173.

In summary, GPR173 is the third CCK receptor, which mediates the CCK signal from CCK-GABA neurons and induces iLTP in their synapses. It is the homologous function of CCK2R that mediates the CCK signal from excitatory CCK neurons and induces the potentiation of excitatory synapses, or LTP (see Li, X., et al., 2014; Chen, X., et al., 2019 cited in Example 3; and Zhang, Z., et al., J. Neurosci. Vol. 40, pp. 2025-2037, 2020).

### Example 9

### Chronic Epileptic Mouse Model Induced after Intrahippocampal Kainic Acid (KA) Injection

Abnormal E/I ratio in the brain would induce several brain disorders, such as autism spectrum disorders (ASD) and schizophrenia spectrum disorders (SSD) (Sohal, V. S., et al., Mol. Psychiatry, Vol. 24, pp. 1248-1257, 2019; Canitano, R., et al., Front. Psychiatry, Vol. 8, pp. 1-7, 2017), and epilepsy (Wiechert, P., et al., J. Neurochem, Vol. 13, pp. 59-64, 1966). Summarizing all the results described above, GPR173 mediates CCK signaling of the CCK-GABA synapse and the potentiation of the inhibition. Could GPR173 be a target to bring back the excitatory/inhibitory (E/I) balance?

This example aims at treating chronic TLE of the mouse model. An intrahippocampal KA-injection induced TLE mouse model using C57BL/6J male mice is adopted, which is a well-established model by previous investigators (Gröticke, I., et al., Exp. Neurol., Vol. 213, pp. 71-83, 2008; Bielefeld, P., et al., Front. Neurosci. Vol. 11, pp. 1-9, 2017). To test our hypothesis that the GPR173 is a potential target for therapeutic intervention in epilepsy, each animal's daily spontaneous seizures are monitored (without any intervention) for 16 weeks after KA injection by our mice seizure recognition system (MMRS). Epileptic seizures are classified into five stages according to Racine's scale (see Observation and assessment of epilepsy by mice movement recognition system (MMRS) discussed above). Only stages 3-5 episodes are included in the present data processing. Animals develop recurrent convulsive seizures from the 2^{nd} or 3^{rd} week after the KA injection. Once mice develop the recurrent convulsive seizures of stages 3 to 5 at week 4, they maintain these seizures in the following 13 weeks (5.2±1.52 seizures per day at week 4). Some mice died before the end of the 16-week observation due to the severe seizure attack (compared to week 4 or 5, N=5 mice; one-way ANOVA with Tukey's post hoc test, N.S.). The model simulates the human TLE, including neuropathological alterations, neuronal cell death, and epileptogenesis (Rusina, E., et al., eNeuro, Vol. 8, ENEURO.0337-20,2021). Many GFAP+ cells (a marker for an abnormally increased number of astrocytes) are found in the right hippocampus (748.56±74.88 per mm²) after 16 weeks, indicating neuronal cell death induced by the right intrahippocampal KA-injection (n=10 sections from N=4 mice).

### Example 10

### Chronic Epilepsy Suppressed in the Long Term after Upregulation of GPR173

The inventors hypothesize that up-regulation of GPR173 may facilitate its signaling in potentiating the inhibition, bringing back the E/I balance, and thus treating the above chronic TLE. The GPR173 is up-regulated into the epileptic mice to see whether it would suppress seizures.

A beneficial and effective therapeutic strategy here is recruiting as many enhanced inhibitory synapses as possible in the epileptic mice, which would work as stop hubs during the progression of the hyperexcitability. Hence, the BBB penetrating AAV is adopted to infect the whole brain instead of gene manipulation in limited brain regions. AAV-PHP.eB carrying AAV-CMV-GPR173-eGFP-PHP.eB (hereinafter referred to as CMV-GPR173-eGFP) or its sham control AAV-CMV-eGFP-PHP.eB (or CMV-eGFP) are produced for the therapy.

KA is injected in the CA1 of the hippocampus to establish the epilepsy model (FIG. 6A). The number of seizures is calculated for 24 hours and manually checked using MMRS as discussed above. No decrease is observed in the frequency of epileptic behaviors within 13 weeks from week 4 after KA injection. Seizures of each mouse are recorded for 2 weeks (including the 4^{th} and 5^{th} weeks after KA injection) as the baseline before treatment (FIG. 6B, Before). Then CMV-GPR173-eGFP (40 μL, 5.01 E+12 vg/mL, experimental group) or CMV-eGFP (40 μL, 5.32 E+12 vg/mL, control group) is infused into the transverse venous sinus in each mouse (FIG. 6A). Their epileptic behaviors are monitored for 8 more weeks (FIG. 6B, After).

Significantly reduced seizures are observed in the mice infused with CMV-GPR173-eGFP in the first two-week after the intervention (weeks 6-7 vs. the baseline of week 4-5: 0.74±0.23 vs. 2.08±0.54, P<0.001, N=8, one-way ANOVA with LSD post hoc test), and it is further reduced in the second two-week (weeks 8-9 vs. baseline 0.47±0.12 vs. 2.08±0.54, P<0.0001). It is worth noting that the mice kept at even lower levels of seizures during the period of observation up to week 13 (weeks 10-11 vs. baseline 0.30±0.07 vs. 2.08±0.54, P<0.0001; week 12-13, 0.45±0.11 vs. 2.08±0.54, P<0.0001). In contrast, no change in seizure occurrence is observed in the control group mice (CMV-eGFP) during the first and second 2-weeks after virus treatment (weeks 6-7 vs. the baseline of week 4-5: 2.23±0.54 vs. 2.68±1.07, P=0.726, weeks 8-9 vs. baseline: 3.40±1.14 vs. 2.68±1.07, P=0.650, N=8, one-way ANOVA with LSD post hoc test), but significant increase occurs from week 10 (weeks 10-11 vs. baseline: 6.17±1.50 vs. 2.68±1.07, P=0.107, weeks 12-13 vs. baseline: 6.08±1.39 vs. 2.68±1.07, P=0.096, N=8).

Finally, anatomical analyses of the brain are conducted on mice from the CMV-GPR173-eGFP and CMV-eGFP groups, confirming that the intravenously infused AAV/PHP.eB successfully penetrated the BBB. eGFP is consistently detected in many brain areas, such as the neocortex, olfactory regions, hippocampal formation, and brainstem.

In summary, it can be concluded that the gene therapy of exogenously delivery of GPR173 successfully suppressed chronic TLE in mice in the long term during the observation of 8 weeks.

### Example 11

### GPR173 Expression Upregulated at CCK-GABA Synapses in the Neocortex

In this example, the followings are examined anatomically: 1) whether the CMV-GPR173-eGFP of the invention successfully upregulates the GPR173 expression in the CCK-GABA synapses; 2) whether GPR173 co-localizes with GABA_{A}R; and 3) the quantitative analysis of GPR173 overexpression in the epilepsy treatment experiment.

A mixture of 600 nL CMV-GPR173-eGFP (1.01 E+12 vg/mL) and mDlx-DIO-ChR2-mCherry (2.09 E+12 vg/mL) are injected into the AC of CCK-Cre mice. Another group of CCK-Cre mice receive 600 nL mixture of the sham control CMV-eGFP (1.06 E+12 vg/mL) and mDlx-DIO-ChR2-mCherry (2.09 E+12 vg/mL). An anatomical verification of reporter eGFP, mCherry (CCK-GABA synapses), and GPR173 (endogenous and exogenous) expression is conducted. The triple co-localization of eGFP, mCherry, and GPR173 is demonstrated, indicating the possible inclusion of exogenously introduced GPR173 (FIG. 7A). The number and size of co-labelings of eGFP, mCherry, and GPR173 are examined to verify whether GPR173 is expressed at the desired location. Group data in FIG. 7B shows that CMV-GPR173-eGFP introduces more GPR173 at CCK-GABA synapses than the sham control CMV-eGFP group. The comparison is conducted in the counts and area of the triple co-localizations of region of interest (ROI) between GPR173 and sham groups (GPR173 vs. Sham: counts: 2496.89±173.30 vs. 354.96±83.36 per mm²; area: 5.18±0.32 vs. 2.37±0.12 μm² per mm² of ROI; GPR173: n=9 sections, N=4 mice; Sham: n=9 sections, N=4 mice; one-way ANOVA with Tukey's post hoc test, ****P<0.0001).

The anatomy of intravenously delivered CMV-GPR173-eGFP or the sham control CMV-eGFP animals after the epilepsy treatment experiment is analyzed in FIG. 6. Upregulation of GPR173 is analyzed with triple labelling in the cortex. The co-localized counts (per mm²) of eGFP, GPR173, and GABA_{A}R indicate that the CMV-GPR173-eGFP introduces more exogenous GPR173 (for example, injecting about 35 to 40 μL of vector per mouse) after penetrating the BBB (FIGS. 7C-7D, GPR173 vs. Sham: 1194.19±130.84 vs. 200.32±51.11; GPR173: n=12 sections, N=5 mice; Sham: n=9 sections, N=3 mice; one-way ANOVA with Tukey's post hoc test, ****P<0.0001). The co-localized counts (/mm²) of GPR173 and GABA_{A}R in the FIG. 7E show that the total expression of GPR173 (both endogenous and exogenous) locating with GABA_{A}R is increased (GPR173 vs. Sham: 2838.77±494.93 vs. 1150.33±265.48; GPR173: n=12 sections, N=5 mice; Sham: n=9 sections, N=3 mice; one-way ANOVA with Tukey's post hoc test, ****P<0.0001).

Here, it is concluded that the GPR173 exogenously expressed are located at the CCK-GABA synapses and near GABA_{A}R. The exogenous introduction of GPR173 is supposed to amplify the CCK signaling leading to recruit more GABA_{A}R within CCK-GABA synapses of TLE mice, leading to suppressing seizures.

### Example 12

### No Change in Locomotion and Astrocytic Reactivity in the Neocortex after Gene Therapy

A preliminary investigation of potential gene therapy-induced aversive effects in the behavioral and structure levels is performed. The activity time after virus injection in both groups are compared. No difference in the amount of active time between the CMV-GPR173-eGFP (injecting about 35 to 40 μL of vector per mouse, 5.01 E+12 vg/mL) or the sham control CMV-eGFP groups (injecting about 35 to 40 μL of vector per mouse, 5.32 E+12 vg/mL) is detected (one-way ANOVA with Tukey's post hoc test, N.S.). The immunoactivity against GFAP is performed to examine the possible cytotoxicity introduced by AAV-PHP.eB. GFAP is a marker for the abnormal increase in astrocytes, mainly caused by the cell apoptosis in the brain. The CMV-GPR173-eGFP or the sham control CMV-eGFP virus does not trigger cell death in the cortex. In contrast, fewer GFAP⁺ cells are detected in the right hippocampus of CMV-GPR173-eGFP-injected TLE mice after therapy (group data for staining of GFAP on the 3 groups of TLE mice: KA only, n=32 left cortical sections (Cortex-L), n=16 right hippocampal sections (Hip-R), N=4 mice; KA+GPR173, n=30 left cortical sections, n=15 right hippocampal sections, N=3 mice; KA+Sham, n=28 left cortical sections, n=14 right hippocampal sections, N=4 mice; one-way ANOVA with Tukey's post hoc test, ****P<0.0001, N.S.). Besides, more Camkll+ cells are found in the cortex of CMV-GPR173-eGFP-injected TLE mice, compared with sham control CMV-eGFP or KA only groups (group data for staining of Camkll on the 4 groups of mice: KA+GPR173, n=32 cortical sections, N=4 mice; KA+Sham, n=37 cortical sections, N=4 mice; Saline, n=87 cortical sections, N=10 mice; KA only, n=88 cortical sections, N=8 mice; one-way ANOVA with Tukey's post hoc test, ****P<0.0001, N.S.), indicating that GPR173 rescues Camkll+ neurons from seizure-induced excitotoxicity in the TLE mice.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A recombinant AAV vector comprising a sequence for introducing the expression of G protein-coupled receptor 173 (GPR173) and specifically targeting GPR173 expressing neurons in a brain.

2. The recombinant AAV vector of claim 1, wherein the recombinant AAV vector further comprises PHP.eB.

3. The recombinant AAV vector of claim 1, wherein the recombinant AAV vector further comprises a gene enhancer sequence selected from the group consisting of CMV, Camkll, mDlx, GAD65, GAD67, and a combination thereof.

4. The recombinant AAV vector of claim 1, wherein the GPR173 comprises an amino sequence according to any of SEQ ID NOs. 1-2, or a functionally equivalent sequence with an identity of at least about 80% to any one of SEQ ID NOs. 1-2.

5. A method of restoring the excitatory/inhibitory (E/I) balance in brain by administrating an effective amount of a recombinant AAV vector of claim 1 or a GPR173 agonist to a subject in need thereof.

6. The method of claim 5, wherein the recombinant AAV vector is administrated at a dose of from about 1E+9 vector genomes to about 1E+15 vector genomes, or the GPR173 agonist is administrated at a dose of from about 1 pmol to about 100 mmol.

7. The method of claim 6, wherein the CCK-GABAergic inhibition is potentiated by increasing the GPR173 expression level in neurons by at least about 25%.

8. The method of claim 7, wherein the GPR173 expression is up-regulated in brain areas selected from the group consisting of cortex, hippocampus, amygdala, entorhinal cortex, and a combination thereof.

9. The method of claim 5, wherein the recombinant AAV vector of claim 1 is administrated by an administration method selected from the group consisting of an intramuscular injection, an intravenous injection, an intraperitoneal injection, a subcutaneous injection, orally taken, a spinal injection, an intraocular injection, and a combination thereof.

10. The method of claim 5, wherein the method further comprises the step of administrating one or more GPR173-related therapeutics selected from the group consisting of small peptide, agonist, nanoparticle, antibody, nucleic acid, mRNA, and a combination thereof.

11. The method of claim 5, wherein the method induces long-term potentiation of CCK-GABAergic inhibition of at least about 2 months.

12. A method of prophylaxis and/or therapy of a neurological condition in a subject in need thereof comprising the step of administrating an effective amount of a recombinant AAV vector of claim 1 or a GPR173 agonist to the subject.

13. The method of claim 12, wherein the neurological condition is selected from the group consisting of epilepsy, autism, depression, schizophrenia, Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, a traumatic brain injury, and a combination thereof.

14. The method of claim 12, wherein the recombinant AAV vector of claim 1 is administrated at a dose of from about 1E+9 vector genome to about 1E+15 vector genome, or the GPR173 agonist is administrated at a dose of from about 1 pmol to about 100 mmol.

15. The method of claim 12, wherein the GPR173 agonist is a CCK analogue.

16. The method of claim 12, wherein the recombinant AAV vector of claim 1 or the GPR173 agonist is administered by an administration method selected from the group consisting of an intramuscular injection, an intravenous injection, an intraperitoneal injection, a subcutaneous injection, a spinal injection, an intraocular injection, and a combination thereof.

17. The method of claim 12, wherein the recombinant AAV vector of claim 1 or the GPR173 agonist induces up-regulation of GPR173 expression so that the GPR173 expression level in neurons increases by at least about 25%.

18. The method of claim 17, wherein the GPR173 expression is up-regulated in brain areas selected from the group consisting of cortex, hippocampus, amygdala, entorhinal cortex, and a combination thereof.

19. The method of claim 12, wherein the method induces long-term potentiation of CCK-GABAergic inhibition of at least about 2 months.

20. The method of claim 12, wherein the subject is a human or a non-human mammal.


SEQUENCE LISTING (16 sequences)

SEQ ID NO: 1 - protein, 373 aa, Mus musculus
MANTTGEPEEVSGALSLPSASAYVKLVLLGLIMCVSLAGNAILSLLVLKERALHKAPYYFLLDLCLADGIRSAICFPFVLASVRHGSSWTFSALSCKIVAFMAVLFCFHAAFMLFCISVTRYMAIAHHRFYAKRMTLWTCAAVICMAWTLSVAMAFPPVFDVGTYKFIREEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLRTHAPCWGTGGAPAPREPYCVM

SEQ ID NO: 2 - protein, 373 aa, Homo sapiens
MANTTGEPEEVSGALSPPSASAYVKLVLLGLIMCVSLAGNAILSLLVLKERALHKAPYYFLLDLCLADGIRSAVCFPFVLASVRHGSSWTFSALSCKIVAFMAVLFCFHAAFMLFCISVTRYMAIAHHRFYAKRMTLWTCAAVICMAWTLSVAMAFPPVFDVGTYKFIREEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLRTHAPCWGTGGAPAPREPYCVM

SEQ ID NO: 3 - DNA, 129 bp, Homo sapiens
  source 1..129: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcc

SEQ ID NO: 4 - DNA, 304 bp, Homo sapiens
  source 1..304: mol_type = genomic DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccat

SEQ ID NO: 5 - DNA, 205 bp, Homo sapiens
  source 1..205: mol_type = genomic DNA
ggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 6 - protein, 289 aa, Mus musculus
MTGVFDRRVPSIRSGDFQAPFPTSAAMHHPSQESPTLPESSATDSDYYSPAGAAPHGYCSPTSASYGKALNPYQYQYHGVNGSAAGYPAKAYADYGYASPYHQYGGAYNRVPSATSQPEKEVAEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYPSAASSINSHLPPPGSLQHPLALASGTLY

SEQ ID NO: 7 - protein, 175 aa, Mus musculus
MSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLPGSAALSPRSPALPPVWDVSASAKGVSMPPNSYMPGYSHWYSSPHQDTMQRPQMM

SEQ ID NO: 8 - protein, 289 aa, Homo sapiens
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 9 - protein, 175 aa, Homo sapiens
MSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLQGSAALSPRSPALPPVWDVSASAKGVSMPPNSYMPGYSHWYSSPHQDTMQRPQMM

SEQ ID NO: 10 - protein, 478 aa, Homo sapiens [sequence omitted]

SEQ ID NO: 11 - protein, 666 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPSRKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 12 - protein, 512 aa, Homo sapiens
MASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILEAVLHCHQMGVVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKINNKANVVTSPKENIPTPALEPQTTVIHNPDGNKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIK

SEQ ID NO: 13 - protein, 588 aa, Homo sapiens
MATTATCTRFTDDYQLFEELGKGAFSVVRRCVKKTSTQEYAAKIINTKKLSARDHQKLEREARICRLLKHPNIVRLHDSISEEGFHYLVFDLVTGGELFEDIVAREYYSEADASHCIHQILESVNHIHQHDIVHRDLKPENLLLASKCKGAAVKLADFGLAIEVQGEQQAWFGFAGTPGYLSPEVLRKDPYGKPVDIWACGVILYILLVGYPPFWDEDQHKLYQQIKAGAYDFPSPEWDTVTPEAKNLINQMLTINPAKRITADQALKHPWVCQRSTVASMMHRQETVECLRKFNARRKLKGAILTTMLVSRNFSVGRQSSAPASPAASAAGLAGQAAKSLLNKKSDGGVKKRKSSSSVHLMPQSNNKNSLVSPAQEPAPLQTAMEPQTTVVHNATDGIKGSTESCNTTTEDEDLKAAPLRTGNGSSVPEGRSSRDRTAPSAGMQPQPSLCSSAMRKQEIIKITEQLIEAINNGDFEAYTKICDPGLTSFEPEALGNLVEGMDFHKFYFENLLSKNSKPIHTTILNPHVHVIGEDAACIAYIRLTQYIDGQGRPRTSQSEETRVWHRRDGKWLNVHYHCSGAPAAPLQ

SEQ ID NO: 14 - protein, 588 aa, Homo sapiens
MSSCVSSQPSSNRAAPQDELGGRGSSSSESQKPCEALRGLSSLSIHLGMESFIVVTECEPGCAVDLGLARDRPLEADGQEVPLDTSGSQARPHLSGRKLSLQERSQGGLAAGGSLDMNGRCICPSLPYSPVSSPQSSPRLPRRPTVESHHVSITGMQDCVQLNQYTLKDEIGKGSYGVVKLAYNENDNTYYAMKVLSKKKLIRQAGFPRRPPPRGTRPAPGGCIQPRGPIEQVYQEIAILKKLDHPNVVKLVEVLDDPNEDHLYMVFELVNQGPVMEVPTLKPLSEDQARFYFQDLIKGIEYLHYQKIIHRDIKPSNLLVGEDGHIKIADFGVSNEFKGSDALLSNTVGTPAFMAPESLSETRKIFSGKALDVWAMGVTLYCFVFGQCPFMDERIMCLHSKIKSQALEFPDQPDIAEDLKDLITRMLDKNPESRIVVPEIKLHPWVTRHGAEPLPSEDENCTLVEVTEEEVENSVKHIPSLATVILVKTMIRKRSFGNPFEGSRREERSLSAPGNLLTKKPTRECESLSELKEARQRRQPPGHRPAPRGGGGSALVRGSPCVESCWAPAPGSPARMHPLRPEEAMEPE

SEQ ID NO: 15 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 16 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL